SEQUENCE LISTING (3 sequences)

SEQ ID NO: 1 - protein, 9 aa, Homo sapiens
RVYIHPFHL

SEQ ID NO: 2 - protein, 100 aa, Homo sapiens
KAKAGAGSATLSMAYAGARFVFSLVDAMNGKEGVVECSFVKSQETECTYFSTPLLLGKKGIEKNLGIGKVSSFEEKMISDAIPELKASIKKGEDFVKTLK

SEQ ID NO: 3 - protein, 72 aa, Homo sapiens
KAKAGAGSATLSMAYAGARFVFSLVDAMNGKEGVVECSFVKSQETECTYFSTPLLLGKKGIEKNLGIGKVSS

(19) United States
(12) Patent Application Publication
(10) Pub. No.: US 2007/0037739 A1
(43) Pub. Date: Feb. 15, 2007

(54) Compounds useful in coating stents to prevent and treat stenosis and restenosis

(75) Inventors: Yuqiang Wang, Cupertino, CA (US); James W. Larrick, Woodside, CA (US); Susan C. Wright, Saratoga, CA (US)

Correspondence Address: PRESTON GATES & ELLIS LLP;ATTN: C. RACHAL WINGER, 925 FOURTH AVE, SUITE 2900, SEATTLE, WA, 98104-1158, US

(73) Assignee: Medlogics Device Corporation, Santa Rosa, CA (US)

(21) Appl. No.: 10/544,241

(22) PCT Filed: Feb. 3, 2004
(86) PCT No.: PCT/US04/03143

§ 371 Date: Jan. 3, 2006

Related U.S. Application Data
(60) Provisional application No. 60/444,391, filed on Feb. 3, 2003.

(51) Int. Cl. A61K 38/17; A61K 31/7034; A61K 31/4745; A61K 31/05

(52) U.S. Cl. 514/12; 514/35; 514/283; 514/44; 514/733

(57) ABSTRACT

At least one bioactive agent is locally delivered to a location where a stent is implanted within a lumen in a patient's body. The bioactive agent includes a: DNA minor groove binder (such as CC-1065 or Duocarmycin); apocynin; RGD peptide (such as RGDfV); stilbene compound (such as resveratrol); camptothecin; des-aspartate angiotensin I; or ADF; or an analog or derivative thereof; or a combination or blend thereof with at least one other bioactive agent. The bioactive agent is generally locally delivered, such as by elution from the stent. The compounds and methods are of particular benefit for treating or preventing atherosclerosis, stenosis, restenosis, smooth muscle cell proliferation, occlusive disease, or other abnormal lumenal cellular proliferation condition.

### CROSS-REFERENCE TO RELATED APPLICATIONS

This application claims priority from U.S. provisional application Ser. No. 60/444,391 filed on Feb. 3, 2003, incorporated herein in its entirety by reference.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### BACKGROUND OF THE INVENTION

1. Field of the Invention

This invention provides bioactive compounds and related systems and methods of manufacture and use that combine the compounds with medical device implants. More specifically, the invention combines local therapy with such compounds at the site of implanted stents.

2. Description of Related Art

Arteries that supply blood and oxygen to the heart muscles are called coronary arteries. Coronary artery disease (CAD) occurs when cholesterol plaque (a hard, thick substance comprised of varying amounts of cholesterol, calcium, muscle cells, and connective tissue, which accumulates locally in the artery walls) builds up in the walls of these arteries, a process called arteriosclerosis. Over time, arteriosclerosis causes significant narrowing of one or more coronary arteries. When coronary arteries narrow more than 50 to 70%, the blood supply beyond the plaque becomes inadequate to meet the increased oxygen demand during exercise. Lack of oxygen (ischemia) in the heart muscle causes chest pain (angina) in most patients. However, some 25% of patients experience no chest pain at all despite documented ischemia, or may only develop episodic shortness of breath instead of chest pain. These patients have silent angina and have the same risk of heart attack as those with angina. When arteries are narrowed in excess of 90-99%, patients often have angina at rest (unstable angina). When a blood clot (thrombus) forms on the plaque, the artery may become completely blocked, causing death of a part of the heart muscles (heart attack, or myocardial infarction).

Angioplasty (also called percutaneous transluminal coronary angioplasty or PTCA) is a general term used to describe a procedure for treating such blockages and/or blood clots. PTCA can produce excellent results in carefully selected patients who may have one or more severely narrowed artery segments, which are suitable for balloon dilatation, stenting, or atherectomy. During PTCA, a local anesthetic is injected into the skin over the artery in the groin or arm. The artery is punctured with a needle and a plastic sheath is placed into the artery. Under x-ray guidance (fluoroscopy), a long, thin plastic tube, called a guiding catheter, is advanced through the sheath to the origin of the coronary artery from the aorta. A contrast dye containing iodine is injected through the guiding catheter so that x-ray images of the coronary arteries can be obtained. A small diameter guide wire (0.014 inches) is threaded through the coronary artery narrowing or blockage. A balloon catheter is then advanced over the guide wire to the site of the obstruction. This balloon is then inflated for about 1 minute, compressing the plaque and enlarging the opening of the coronary artery. Balloon inflation pressures may vary from as little as one or two atmospheres of pressure, to as much as 20 atmospheres. Finally, the balloon is deflated and removed from the body.

Over the last decade, new devices that can cut out pieces of a plaque, vaporize it with a laser, bore out the blockage with a kind of surgical drill bit, or insert a tiny metal, stent, spring into the coronary artery to help keep it stretched open have been developed. After the coronary artery blockage has been treated by angioplasty, a small, expandable metal scaffold (the stent) is inserted into the artery and expanded. The purpose of the stent is to maintain the opening created by the angioplasty, and prevent a recurrence of the blockage. Intracoronary stents are deployed in either a self-expanding fashion, or most commonly they are delivered over a conventional angioplasty balloon. When the balloon is inflated, the stent is expanded and deployed, and the balloon is removed, the stent remains in place in the artery. Atherectomy devices are inserted into the coronary artery over a standard angioplasty guide wire, and then activated in varying fashion, depending on the device chosen.

There are several reasons to undergo an angioplasty procedure. If chest pain symptoms are not easily controlled with medications, or if symptoms prevent the patent from participating in daily activities, an angioplasty may decrease or eliminate the chest pains. After the procedure, fewer cardiac medications may be required. If the patient is experiencing chest pains at rest (i.e., without exercise or exertion), or if chest pain continues after a heart attack, an angioplasty procedure is used to treat the blockage causing the problem. One recently completed study found that in certain male patents with chest pains at rest, including those who had suffered a small heart attack, treatment of coronary stenosis with an angioplasty procedure resulted in fewer long-term adverse events than treatment with medications alone.

Long-term benefits of PTCA depend on the maintenance of the newly-opened coronary artery(ies). Recurrent narrowing (restenosis) of a coronary artery by formation of new blockages at the site of the angioplasty or stent occurs within 3-6 months in 40-50% of patients who have angioplasty. This incidence has been reduced to 20-30% with the use of stents. Obviously, whether a stent is used or not restenosis remains a major problem. There are two major mechanisms for restenosis. The first is by thrombosis, or blood clotting, at the site of treatment. The risk of thrombosis is the greatest immediately after angioplasty, because the resultant tissue trauma tends to trigger blood clotting. This form of restenosis is greatly reduced by using anti-clotting drugs for a time during and after the procedure. The second form of restenosis is tissue growth at the site of treatment. This form of restenosis is a proliferation of the endothelial cells that normally line blood vessels tends to occur during the first 3 to 6 months after the procedure, and is not prevented by anti-clotting drugs.

The clotting mechanism is one of the most important and complex of physiologic systems. Blood must flow freely through the blood vessels in order to sustain life. But if a blood vessel is traumatized, the blood must clot to prevent life from flowing away. Thus, the blood must provide a system that can be activated instantaneously—and that can be contained locally—to stop the flow of blood. This system is called the clotting mechanism.

There are two major facets of the clotting mechanism—the platelets, and the thrombin system. The platelets are tiny cellular elements, made in the bone marrow, that travel in the bloodstream waiting for a bleeding problem to develop. When bleeding occurs, chemical reactions change the surface of the platelet to make it “sticky.” Sticky platelets are “activated.” These activated platelets begin adhering to the wall of the blood vessel at the site of bleeding, and within a few minutes they form what is called a “white clot,” a clump of platelets appears white to the naked eye. The thrombin system consists of several blood proteins that, when bleeding occurs, become activated. The activated clotting proteins engage in a cascade of chemical reactions that finally produce a substance called fibrin. Fibrin can be thought of as a long, sticky string. Fibrin strands stick to the exposed vessel wall, clumping together and forming a web-like complex of strands. Red blood cells become caught up in the web, and a “red clot” forms.

A mature blood clot consists of both platelets and fibrin strands. The strands of fibrin bind the platelets together, and “tighten” the clot to make it stable. In arteries, the primary clotting mechanism depends on platelets. In veins, the primary clotting mechanism depends on the thrombin system. But in reality, both platelets and thrombin are involved, to one degree or another, in all blood clotting.

The clotting system, like all complex physiologic systems, can produce problems. Blood clots forming on atherosclerotic plaques in the arteries are the major cause of heart attack and stroke. Blood clots forming in the veins of the legs produce a painful condition called phlebitis, and when these venous blood clots break off (“embolize”) they move into the lungs and produce a dangerous condition called pulmonary embolus.

Drugs are used to prevent or treat abnormal blood clotting. These drugs can be aimed either at the platelets, or at the thrombin system.

Drugs aimed at the thrombin system.

Certain drugs prevent further fibrin from forming. These drugs, which inhibit one or more of the proteins involved in the thrombin clotting system, are used for both arterial and venous clotting problems. Certain examples of these drugs follow.

Heparin. Heparin is an intravenous drug that has an immediate (within seconds) inhibitory effect on the thrombin system. Its dosage can be adjusted frequently, following the PTT blood test (the partial thromboplastin time) to achieve the desired effect.

Low molecular weight heparin: enoxaparin, dalteparin. LMWH is a “purified” derivative of heparin. Its major advantages are that it can be given as a skin injection (which almost anyone can learn to do in a few minutes), and does not need to be closely monitored with blood tests. Thus, unlike heparin, LMWH can be administered safely on an outpatient basis.

Coumadin: Coumadin is an oral anti-thrombin drug that can be taken chronically. The dose must be carefully monitored by following the prothrombin time (PT), a blood test

Other drugs are adapted to instead “dissolve” fibrin—otherwise generally referred to as fibrinolytic drugs. These powerful drugs actually dissolve fibrin strands that have already formed. Certain examples of these types of drugs follow immediately below.

TPA, streptokinase, urokinase. These are the intravenous drugs that are administered acutely during the first few hours of an acute heart attack or stroke, to attempt to re-open an occluded artery, and prevent permanent tissue damage.

Drugs aimed at platelets.

These three groups of drugs, in one way or another, reduce the “stickiness” of platelets. They are used most commonly in preventing arterial clots from forming. Examples include the following.

Aspirin and diypyramidole. These drugs have a modest effect on platelet “stickiness,” but have few important side effects.

Ticlopidine (TicIId) and clopidrogel (Plavix). These drugs are somewhat more powerful than the first group, but can be poorly tolerated and can have important side effects. They are generally used in patients who need, but cannot tolerate, aspirin.

IIb/IIIa inhibitors: abciximab (Reopno), eptifabitide (Integrilin), tirofiban (Aggrastat). The IIb/IIIa inhibitors are the most powerful group of platelet inhibitors. They inhibit a receptor on the surface of platelets (the so-called IIb/IIIa receptor) that is essential for platelet stickiness. Their chief usage is to prevent acute clotting after interventional procedures (such as angioplasty and stent placement), and in patients with acute coronary artery syndromes, such as unstable angina. These drugs are very expensive and (in general) must be given intravenously.

The most immediate threat of restenosis, especially after stent placement, is thrombosis. For several years, clinical trials have been conducted to devise methods of reducing this form of restenosis. It has now been learned that administering special anti-platelet drugs called IIb/IIa inhibitors (i.e., the drugs abciximab and eptifabatide) significantly diminish this problem. Thus, tissue growth (i.e., the scar-like) restenosis is the major remaining problem.

Solving tissue growth restenosis has proven to be a tall order. To date, the most effective method of reducing the risk of restenosis has been the use of stents. In fact, the major advantage of stents over angioplasty alone is that with stents the incidence of restenosis has been significantly reduced. However, the risk of restenosis during the first 6 months after a stent remains as high as 20-30%. One of the hottest areas of biomedical research today is in devising stents that inhibit restenosis. A molecular approach is a highly beneficial solution for the restenosis problem (Sousa et al. Circ 2003; 107:2274-2279). The approach with the most immediate promise, and accomplishments toward this goal, is to make drug-coated stents. These stents are coated with drugs that inhibit the tissue growth that causes restenosis. Many drugs can inhibit the growth of cells. While many of them would be considered too risky to administer throughout the entire body, the idea of delivering a tiny amount of the drug directly to the tissue that needs to be inhibited is a very attractive one.

Several drug-coated stents have been the topic of clinical trials in Europe and the United States. The most commonly mentioned are sirolimus-coated stents, rapamycin-coated stents, and paclitaxel-coated stents. In addition, a new technique has been developed to coat stents with a polymer that can deliver DNA to the local tissue. While stent-delivered DNA therapy to inhibit restenosis is farther off than therapy with drug-coated stents, it also has a lot of potential.

The first drug-coated stent has been approved for marketing in Europe. The Johnson & Johnson sirolimus-coated stent (brand name: Cypher) was quickly approved after results from the RAVEL trial were presented. The RAVEL trial confirmed the remarkable early finding that there were no instances of restenosis in patients receiving the sirolimus stent. The Cypher stent has been priced as much as 200-400% higher than non-coated stents, so cost is a concern to European hospitals and health care systems. But investigators in the RAVEL trial maintain that their data shows that when one factors in the cost savings produced by eliminating restenosis (not to mention the morbidity to the patients that is avoided,) using the drug-coated stent is actually cost-effective.

The results of two large clinical trials using drug-coated stents were also presented at the Transcatheter Cardiovascular Therapeutics 2002 scientific sessions in Washington D.C. The first of the two trials, the SIRIUS trial, examined the use of the sirolimus-coated stent, from Cordis and Johnson & Johnson. Previous trials with the sirolimus-coated stent suggested a remarkable reduction in restenosis compared to using “bare” metal stents. However, the earlier trials were largely limited to patients whose coronary artery blockages were considered nearly ideal for the use of stents. In the SIRIUS trial, in contrast, patients were intentionally enrolled whose blockages were considered high-risk. Despite this higher risk population of patients, the SIRIUS trial showed a pronounced reduction in the rate of restenosis among patients receiving the sirolimus-coated stents. Patients receiving the drug-coated stent had a 91% reduction in restenosis within the stent itself. The main endpoint of the study, however, was not restenosis but “target vessel failure” defined as cardiac death, heart attack, or the need for revascularization within 9 months of stent placement. The drug-coated stents reduced target vessel failure from 21% to 8.6%. The CYPHER™ DES stent that was the subject of these and subsequent trials has been approved for sale in the United States, in addition to Europe.

In the second trial, TAXUS II, results with a paclitaxel-coated stent from Boston Scientific were presented. Overall results were comparable to those achieved with the sirolimus-coated stents. The related TAXUS™ DES product has been approved for sales in Europe.

Both the SIRIUS and TAXUS trials have been further expanded to additional patent populations, with generally positive results.

Accordingly, at least two types of drug-coated stents continue to yield remarkable decreases in the rate of restenosis when compared to standard, bare-metal stents. However, though at substantially improved rates, restenosis still occurs for many patients receiving DES implants coated with these drugs. Such rates generally range from about 5% to about 9% in the overall population, in other sub-groups, such as cases of “bifurcation” stenting or diabetics, the rate is higher for one or both of these approaches. In addition, stent strut “malapposition”, or separation between the stent strut and the vessel wall has been observed in some DES implants. These have been associated by some as a result of “pseudoaneurysm” formation, which is further believed to relate to certain toxic side effects of the chosen drugs in the vessel wall. Both Rapamycin (sirolimus) and paclitaxel are generally considered toxic compounds, previously used to kill tumor cells or as immunosuppresants to prevent organ transplant rejection. As antimitotic and antproliferative foreign compounds, and proper dosing is imperative to avoid unwanted toxicity. In the event such toxicity is experienced in the vessel wall, it is believed the wall may respond by weakening or withdrawing outwardly from the stent itself as the toxic source.

In general, despite recent successes and improvements, a need still exists for improved local drug therapies for treating or preventing atherosclerosis, stenosis, restenosis, smooth muscle cell proliferation, occlusive disease, or other abnormal lumenal cellular proliferation conditions.

### BRIEF SUMMARY OF THE INVENTION

Accordingly, various aspects, modes, embodiments, variations, and features of the invention are described as follows.

One aspect of the invention is a system for providing therapy to a region of tissue associated with a lumen in a patient. This system includes an endolumenal stent that is adapted to be implanted at a location within a lumen associated with the region of tissue, a local delivery system, and a bioactive agent. The local delivery system is adapted to locally deliver the bioactive agent to the location, and the bioactive agent when locally delivered to the location is adapted to treat the medical condition. The bioactive agent comprises at least one of: CC-1065, duocarmycin, apocynin, RGDfV, RGD peptide, resveratrol, stilbene, camptothecin, DAA-1, or ADF, or an analog or derivative thereof, or a pharmaceutically acceptable salt thereof, or a combination or blend thereof.

Another aspect is a system for treating or preventing atherosclerosis, stenosis, restenosis, smooth muscle cell proliferation, occlusive disease, or other abnormal lumenal cellular proliferation condition providing interventional medical care to a patient. This system includes a local delivery system in combination with a bioactive agent as follows. The local delivery system is adapted to locally deliver the bioactive agent to a region of tissue associated with the condition. The bioactive agent when locally delivered to the region of tissue is adapted to treat or prevent the condition, and in particular comprises at least one of CC-1065, duocarmycin, apocynin, RGDfV, RGD peptide, resveratrol, a stilbene compound, camptothecin, des-aspartate angiotensin I (“DAA-1”), or apoptosis DNA factor (“ADF”), or an analog or derivative thereof, or a pharmaceutically acceptable salt thereof, or a combination or blend thereof.

According to one mode of this aspect, the bioactive agent comprises CC-1065 or an analog or derivative thereof, or a pharmaceutically acceptable salt thereof.

According to another mode, the bioactive agent comprises duocarmycin or an analog or derivative thereof, or a pharmaceutically acceptable salt thereof.

According to another mode, the bioactive agent comprises apocynin or an analog or derivative thereof, or a pharmaceutically acceptable salt thereof.

According to another mode, the bioactive agent comprises RGDfV or an analog or derivative thereof, or a pharmaceutically acceptable salt thereof.

According to another mode, the bioactive agent comprises an RGD peptide or an analog or derivative thereof, or a pharmaceutically acceptable salt thereof.

According to another mode, the bioactive agent comprises resveratrol or an analog or derivative thereof, or a pharmaceutically acceptable salt thereof.

According to another mode, the bioactive agent comprises a stilbene compound or an analog or derivative thereof, or a pharmaceutically acceptable salt thereof.

According to another mode, the bioactive agent comprises camptothecin or an analog or derivative thereof, or a pharmaceutically acceptable salt thereof.

According to another mode, the bioactive agent comprises DAA-1 or an analog or derivative thereof, or a pharmaceutically acceptable salt thereof.

According to another mode, the bioactive agent comprises ADF or an analog or derivative thereof, or a pharmaceutically acceptable salt thereof.

According to another mode, the system further includes an interventional device adapted to perform a medical procedure at or adjacent to the location of the local drug delivery.

According to one embodiment of this mode, the interventional device is a stent.

According to one further embodiment, the local delivery system comprises a drug release vehicle associated with the stent.

According to yet a further embodiment, the drug release vehicle is a coating on the stent.

In one variation of this embodiment, the coating comprises a polymer.

Another aspect of the invention is a method for treating or preventing atherosclerosis, stenosis, restenosis, smooth muscle cell proliferation, occlusive disease, or other abnormal lumenal cellular proliferation condition within a body of a patient. This method includes locally delivering a bioactive agent at a location within the patent's body in a manner that is adapted to substantially treat or prevent the atherosclerosis, stenosis, restenosis, smooth muscle cell proliferation, occlusive disease, or other abnormal lumenal cellular proliferation condition. The bioactive agent used according to this method includes at least one of CC-1065, duocarmycin, apocynin, RGDfV, RGD peptide, resveratrol, a stilbene compound, camptothecin, des-aspartate angiotensin I (“DAA-1”), or apoptosis DNA factor (“ADF”), or an analog or derivative thereof, or a pharmaceutically acceptable salt thereof, or a combination or blend thereof.

In one further mode of this aspect, the method further includes injuring a wall of a lumen in the patient's body, and wherein the bioactive agent is locally delivered to the location in a manner adapted to substantially treat or prevent restenosis associated with the wall injury.

Another mode includes implanting a stent at the location. One further embodiment of this mode further includes beneficially eluting the bioactive agent from the stent at the location.

It is to be appreciated that each of the various aspects, modes, embodiments, and variations just described is independently beneficial and without requiring combination with the others. Nevertheless, it is further understood that the various combinations and sub-combinations thereof also constitute further beneficial aspects hereof, as would be apparent to one of ordinary skill based upon review of the totality of this disclosure in combination with other available information.

It is to be appreciated that the various compound delivery aspects and related systems and methods of the various modes and embodiments may be accomplished according to further aspects for treating or preventing other tissue conditions adjacent to luminal wall structures, such as for local therapy or prophylaxis of inflammation or cancer adjacent to stented vessels or other body spaces.

Further aspects of the invention will be brought out in the following portions of the specification, wherein the detailed description is for the purpose of fully disclosing preferred embodiments of the invention without placing limitations thereon.

### BRIEF DESCRIPTION OF THE DRAWINGS

The invention will be more fully understood by reference to the following drawings which are for illustrative purposes only:

FIG. 1 shows schematic views of certain molecules for use according to various embodiments of one aspect of the invention.

FIG. 2 shows a schematic flow diagram of a particular scheme for synthesizing certain molecules according to certain embodiments of the invention shown in FIG. 1.

FIG. 3 shows a biochemical pathway related to the embodiment of the invention shown in FIG. 1.

FIG. 4 shows a schematic view of another molecule illustrative of a further embodiment for use according to certain aspects of the invention.

FIG. 5 shows a schematic view of another molecule illustrative of a further embodiment for use according to certain aspects of the invention.

FIGS. 6A-B show various molecules illustrative of further embodiments for use according to certain aspects of the invention.

FIG. 7A shows a graph demonstrating restenosis results of a pre-clinical animal study comparing a control vehicle group versus various treatment groups receiving systemic doses of different concentrations of another anti-restenosis compound useful according to a further aspect of the invention.

FIG. 7B shows cross-sectioned histological pictures of control arteries (top) and an artery from one of the treatment groups (bottom) according to the same experiment that formed the basis for the graph in FIG. 7A.

FIGS. 8A-B show graphs demonstrating certain effects of the compound related to the results shown FIGS. 7A-B, but with respect to Angiotensin II stimulated MAP Kinase activity in vascular smooth muscle cells and cardiomyocytes, respectively.

FIG. 9 shows various molecules that represent further embodiments for use according to one or more aspects described herein.

FIG. 10 shows a schematic flow diagram of an illustrative medical procedure according to one aspect of the invention.

FIG. 11 shows a stented region of an artery according to one mode of the invention useful for example according to the aspect shown in FIG. 10.

FIG. 12 shows a cross section of a stent strut coated with a bioactive agent according to a further aspect of the invention and useful for example according to the aspects illustrated in FIGS. 10 and 11.

### DETAILED DESCRIPTION OF THE INVENTION

It is to be appreciated therefore that certain aspects, modes, embodiments, variations and features of the invention described below in various levels of detail in order to provide a substantial understanding of the present invention. In general, such disclosure provides beneficial compounds, combinations of such compounds with other devices, assemblies, and systems, and related methods. Such are generally considered well adapted to enhance the treat or inhibit stenosis, or restenosis, or are otherwise provided in combination with implantable stents.

Accordingly, the various aspects of the present invention relate to therapeutic uses of certain particular bioactive agents or compounds for local delivery in combination with stents or other recanalization therapies in order to prevent or treat restenosis. Accordingly, various particular embodiments that illustrate these aspects follow.

It is to be appreciated that the various modes of treatment or prevention of medical conditions as described are intended to mean “substantial”, which includes total but also less than total treatment or prevention, and wherein some biologically or medically relevant result is achieved.

Definitions

“Basic amino acid,” as used herein, refers to a hydrophilic amino acid having a side chain pK value of greater than 7. Basic amino acids typically have positively charged side chains at physiological pH due to association with hydronium ion. Examples of genetically encoded basic amino acids include arginine, lysine and histidine. Examples of non-genetically encoded basic amino acids include the non-cyclic amino acids ornithine, 2,3-diaminopropionic acid, 2,4-diaminobutyric acid and homoarginine.

A “subject,” as used herein, is preferably a mammal, such as a human, but can also be an animal, e.g., domestic animals (e.g., dogs, cats and the like), farm animals (e.g., cows, sheep, pigs, horses and the like) and laboratory animals (e.g., rats, mice, guinea pigs and the like).

An “effective amount” of a compound, as used herein, is a quantity sufficient to achieve a desired therapeutic and/or prophylactic effect, for example, an amount which results in the prevention of or a decrease in the symptoms associated with a disease that is being treated, e.g., the diseases associated with TGF-beta superfamily polypeptides listed above. The amount of compound administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. Typically, an effective amount of the compounds of the present invention or polynucleotides encoding the compounds of the present invention, sufficient for achieving a therapeutic or prophylactic effect, range from about 0.000001 mg per kilogram body weight per day to about 10,000 mg per kilogram body weight per day. Preferably, the dosage ranges are from about 0.0001 mg per kilogram body weight per day to about 100 mg per kilogram body weight per day. The compounds of the present invention can also be administered in combination with each other, or with one or more additional therapeutic compounds.

The term “variant,” as used herein, refers to a compound that differs from the compound of the present invention, but retains essential properties thereof. A non-limiting example of this is a polynucleotide or polypeptide compound having conservative substitutions with respect to the reference compound commonly known as degenerate variants. Another non-limiting example of a variant is a compound that is structurally different, but retains the same active domain of the compounds of the present invention, for example, N-terminal or C-terminal extensions or truncations of a polypeptide compound. Generally, variants are overall closely similar, and in many regions, identical to the compounds of the present invention. Accordingly, the variants may contain alterations in the coding regions, non-coding regions, or both.

The term “sequence identity,” as used herein, refers to the degree to which two polynucleotide or polypeptide sequences are identical on a residue-by-residue basis over a particular region of comparison.

The term “percentage of sequence identity,” as used herein, is calculated by comparing two optimally aligned sequences over that region of comparison, determining the number of positions at which the identical amino acids occurs in both sequences to yield the number of matched positions, dividing the number of matched positions by the total number of positions in the region of comparison (i.e., the window size), and multiplying the result by 100 to yield the percentage of sequence identity.

The term “substantial identity,” as used herein, denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Sequence identity can be measured using sequence analysis software (Sequence Analysis Software Package of the Genetics Computer Group, University of Wisconsin Biotechnology Center, 1710 University Avenue, Madison, Wis. 53705), with the default parameters therein.

In the case of polypeptide sequences, which are less than 100% identical to a reference sequence, the non-identical positions are preferably, but not necessarily, conservative substitutions for the reference sequence. Conservative substitutions typically include substitutions within the following groups: glycine and alanine; valine, isoleucine, and leucine; aspartic acid and glutamic acid; asparagine and glutamine; serine and threonine; lysine and arginine; and phenylalanine and tyrosine. Thus, included in the invention are peptides having mutated sequences such that they remain homologous, e.g., in sequence, in structure, in function, and in antigenic character or other function, with a polypeptide having the corresponding parent sequence. Such mutations can, for example, be mutations involving conservative amino acid changes, e.g., changes between amino acids of broadly similar molecular properties. For example, interchanges within the aliphatic group alanine, valine, leucine and isoleucine can be considered as conservative. Sometimes substitution of glycine for one of these can also be considered conservative. Other conservative interchanges include those within the aliphatic group aspartate and glutamate; within the amide group asparagine and glutamine; within the hydroxyl group serine and threonine; within the aromatic group phenylalanine, tyrosine and tryptophan; within the basic group lysine, arginine and histidine; and within the sulfur-containing group methionine and cysteine. Sometimes substitution within the group methionine and leucine can also be considered conservative. Preferred conservative substitution groups are aspartate-glutamate; asparagine-glutamine; valine-leucine-isoleucine; alanine-valine; phenylalanine-tyrosine; and lysine-arginine.

The invention also provides for compounds having altered sequences including insertions such that the overall amino acid sequence is lengthened, while the compound still retains the appropriate smooth muscle cell modulating property, e.g., inhibition of the cellular activation of smooth muscle, e.g., but not limited to, phosphorylation of retinoblasoma protein (pRp), modulation of p27kip1 protein, and binding of target molecule(s), that can lead to smooth muscle cell proliferation. Preferably, conservative amino acid substitutions are those wherein an amino acid is replaced with another amino acid encompassed within the same designated class, as will be described more thoroughly below. Insertions, deletions, and substitutions are appropriate where they do not abrogate the functional properties of the compound. Functionality of the altered compound can be assayed according to the in vitro and in vivo assays described below that are designed to assess the properties of the altered compound.

The references cited throughout this application are incorporated herein by reference in their entireties.

Compositions of the Present Invention

Apocynin and Certain Derivatives

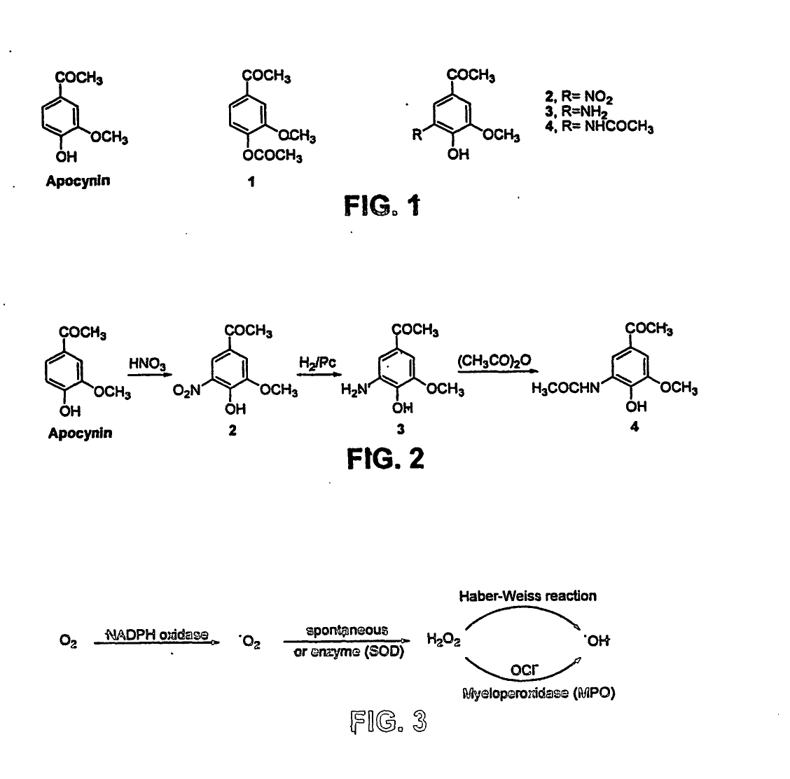

Apocynin is a particularly beneficial compound that is naturally occurring and well known ant-Inflammatory supplement as a Chinese herbal remedy. Apocynin is generally represented by the molecule shown on the left side of FIG. 1. As described in further detail below, apocynin itself is considered a highly beneficial embodiment for use according to various of the aspects described herein. In addition, other modifications are contemplated have been synthesized in order to enhance or otherwise alter certain desired biological activities or other characteristics of the compound, such as for example according to the additional molecules variously labeled 1-4, also in FIG. 1. The synthesis of these compounds is described for illustration as follows according to their respective labels and parenthetic designation as molecules (1)-(4) by reference to FIG. 1. These derivatives (e.g., nitrosylated apocynin) represent novel compositions.

4-Acetoxy-3-methoxyacetophenone (1). To a solution of apocynin (2 g, 12 mmol) in 20 ml of ethyl acetate cooled to 0° C. was added acetyl chloride (1.28 ml, 18 mmol) followed by triethylamine (2.5 ml, 18 mmol) dropwise under nitrogen. The reaction mixture was allowed to warm to room temperature and was stirred for 2 h. The mixture was washed with water (50 ml×3), and the organic layer was dried using sodium sulfate. The solvent was removed, and the product was crystallized in ethyl ether to afford 1.8 g (72% yield) of white crystals. mp 56-57° C. ¹H NMR (acetone-d₆, ppm): 7.67-7.56 (m, 2H, Ar—H), 7.21-7.13 (d, 1H, Ar—H), 3.88 (s, 3H, OCH₃), 2.57 (s, 3H, COCH₃), 2.27 (s, 3H, O COCH₃), MS 208.

Compounds 2-4 were synthesized as shown in general overview in Scheme 1 in FIG. 2. Further detail variously related to the synthesis of these compounds is also described as follows.

4-Hydroxy-3-methoxy-5-nitroacetophenone (2). Concentrated nitric acid (70%, 16.3 ml) was added dropwise to a solution of apocynin (10.2 g, 61 mmol) in 500 ml of chloroform at 0° C., and the solution was stirred for an additional 2 h. The reaction mixture was washed with water (70 ml×7), and the organic layer was dried using sodium sulfate. Solvent was removed in vacuo, and 250 ml of 95% ethanol was added. The product was crystallized overnight to afford 9.5 g (73% yield) of product as yellow needles, mp 159-161° C. ¹H NMR (CDCl₃, ppm): 11.13 (s, 1H, OH), 8.32-8.31 (d, 1H, Ar—H), 7.78-7.77 (d, 1H, Ar—H), 4.02 (s, 3H, OCH₃), 2.64 (s, 3H, COCH₃), MS 211.

5-Amino-4-hydroxy-3-methoxyacetophenone hydrochloride (3). To a solution of 2 (2 g, 9.5 mmol) in ethyl acetate was added 10% Pd/C (200 mg), and the reaction mixture was hydrogenated for 1 h at a pressure of 40 lb/inch². The reaction mixture was filtered, and solvent was removed. Concentrated HCl was added, and the precipitate was filtered. The product was then crystallized in ethanol to afford 1.1 g (53% yield) of product as colorless needles. ¹H NMR (D₂O, ppm): 7.57-7.55 (d, 1H, Ar—H), 7.45-7.43 (d, 1H, Ar—H), 3.88 (s, 3H, OCH₃), 2.55 (s, 3H, COCH₃), MS 181.

5-Acetoamido-4-hydroxy-3-methoxyacetophenone (4). To a solution of 2 (1.5 g, 9.0 mmol) in ethyl acetate was added 10% Pd/C (150 mg), and the reaction mixture was hydrogenated for 1 h at a pressure of 40 lb/inch². The reaction mixture was filtered. Without further purification, the filtrate was cooled to 0° C., and acetic anhydride (1.0 ml, 10.8 mmol) and dimethylaminopyridine (5 mg) were added. The reaction mixture was stirred at room temperature for 30 min, and solvent was removed. The product was crystallized in THF and petroleum ether to afford 1.3 g (65% yield for two steps) of product as brown-crystals, mp 180-181° C. ¹H NMR (DMSO-d₆, ppm): 10.02 (brs, 1H, NH), 9.38 (s, 1H, OH), 8.07 (d, 1H, Ar—H), 7.30 (s, 1H, Ar—H), 3.87 (s, 3H, OCH₃), 2.48 (s, 3H, COCH₃), 2.11 (s, 3H, NHCOCH₃), MS 223.

Further information related to apocynin, including certain characteristics and observed bioactivities, and further related to certain of the derivatives described herein, is provided for illustration as follows. Such description is provided together with citations to certain available publications for the purpose of further reference, which publications if previously cited herein are only cited in partial form.

Apocynin is the major active component of Picrorhiza kurroa, one of the most popular herbs used by the Chinese for centuries to treat diseases connected with inflammation. (Bensky D and Gamble A. (eds.) 1986 Chinese Herbal Medicine Materia Medica, Seattle: Eastland Press., pp. 120-121). Cytokines and reactive oxygen species (ROS) play a central role in the pathogenesis of rheumatoid arthritis (RA).

Rheumatoid arthritis (RA) is a major medical problem affecting up to 3% of the population in many countries and about 2.5 million people in the United States. RA is a chronic destructive inflammatory disease affecting the synovial membrane and extra-articular tissues. Inflammatory particles accumulate and persist in the synovial membrane, leading to destruction of joint architecture. (Weyand, C. M.; Goronzy, J. J. The molecular basis of rheumatoid arthritis, J. Mol. Med. 1997, 75, 772-785). The ultimate consequences of RA are significant levels of pain, Immobility, functional disability, and rheumatoid organ involvement. Although the cause of RA is not understood completely today, K is known that the development of RA is mediated by a number of cellular and molecular components, which include both the oxygen- and nitrogen-containing ROS and certain cytokines such as tumor necrosis factor alpha (TNF-α) and interleukin-1 (IL-1) (Bondeson, J. The mechanisms of action of disease-modifying antirheumatic drugs: A review with emphasis on macrophage signal transduction and the induction of proinflammatory cytokines. Gen. Pharmacol, 1997, 29, 127-150; Bauerova, K., Bezek, S. Role of reactive oxygen and nitrogen species in etiopathogenesis of rheumatic arthritis, Gen. Physiol. Biophys. 1999, 18, 15-20; Weyand and Goronzy, 1997).

Neutrophils are one of the two classes of white blood cells that act as professional phagocytes to defend against acute bacterial, fungal and other foreign infections. Neutrophils kill previously opsonized microorganisms by reactive oxygen species (ROS). ROS are mainly generated in a sequential manner during oxidative bursts by the activation of the neutrophil membrane-bound NADPH oxidase in response to a wide range of stimuli including the chemotactic peptide FLMP, the complement component C5a, various cytokines such as TNF-α. IL-1, and opsonized particles (Babior. B. M.; Kipnes, R. S.; Curnutte, J. T. Biological defense mechanisms. The production by leucocytes of superoxide, a potent bactericidal agent. J. Clin. Invest. 1973, 52, 741-744; Rossi, F. The O₂-forming NADPH oxidase of the phagocytes: nature, mechanisms of activation and function. Biochim. Biophy. Acta, 1986, 853, 65-89; Cross, C. E. Oxygen radicals and human disease. Ann. Intern. Med. 1987, 107, 526-545; Bellavite, P. The superoxide-forming enzymatic system of phagocytes. Free Radic. Biol. Med. 1988, 4, 225-261).

Initially, superoxide anion (O₂) is formed by the one-electron reduction of free molecular oxygen by NADPH oxidase (FIG. 3). O₂ is converted to hydrogen peroxide (H₂O₂) either spontaneously or enzyme-dependently, and the latter is converted to the highly reactive hydroxy-radical (OH) through the Haber-Weiss reaction or in the presence of halogen anions via hypohalides (e.g., OCl⁻) in a reaction governed by myeloperoxidase (MPO) (Fantone, J. C. and Ward, P. A. Am. J. Pathol. 1982, 107, 397-417). Whereas superoxide and hydrogen peroxide are cellular signals that initiate the expression of pro-inflammatory cytokines, singlet oxygen and hydroxy radicals are very reactive and can oxidize various important biological molecules including DNA, protein, membrane lipid, and extracellular matrix such as collagen.

The pro-inflammatory cytokines TNF-α and IL-1 produced in large amounts by inflamed synovial membranes play a pivotal role in the acute phase of RA (Beutler, B.; Cerami, A. The biology of cachectin/TNF: a primary mediator of the host response. Annu. Rev. Immunol. 1989, 7, 625-655; Beutler, B. Tumor necrosis factor In: The molecules and their emerging role in medicine. Raven Press, New York. 1992; Tetta, C.; Camussi, L; Modena, V.; Di Vittoria, C.; Baglioni, C. Tumor necrosis factor in serum and synovial fluid of patients with active and severe rheumatoid arthritis. Ann Rheum. Dis. 1990, 49, 665-667). There is a correlation between the number of mononuclear phagocytes and the level of TNF-α and IL-1 production (Bondeson, 1997). TNF-α is a powerful inducer of NADPH oxidase activity. It enhances the assembly process of phagocytic NADPH oxidase to the active enzyme by inducing the expression of important regulatory sub-units, thereby maintaining the enzyme in an activated state (Gupta, J. W.; Kubi, M.; Hartman, L.; Casatella, M.; Trinchieri, G. Induction of expression of genes encoding components of the respiratory burst oxidase during differentiation of human myeloid cell lines induced by tumor necrosis factor and gamma-interferon. Cancer Res. 1992, 52, 2530-2537; Utsumi, T. J.; Klostergaard, K.; Akimaru, K.; Edashige, E. F.; Sato, L.; Utsumi, K. Modulation of TNF-alpha-priming and stimulation-dependent superoxide generation in human neutrophils by protein kinase inhibitor. Arch. Biochem. Biophys. 1992, 294, 271-278). ROS activate the cytosolic transcription of nuclear factor kappa B (NF-κB) (Schreck, R.; Albermann, K.; Baeuerle, P. A. Nuclear factor κB: an oxidative stress-response transcription factor of eukaryotic cells [a review], Free Radic. Res. Commun. 1992, 17, 221-237). The later induces the expression of the TNF-α gene amongst other genes (Lenardo, M. J.; and Baltimore, D. NF-κB: a pleiotropic mediator of inducible and tissue-specific gene control. Cell, 1989, 58, 227-229). The increased production of TNF-α causes further activation of NADPH oxidase (Lenardo and Baltimore, 1989). Thus a positive feedback loop may form, in which ROS induce NF-κB-dependent TNF-α expression, which further activates phagocytic NADPH oxidases leading to the production of more ROS.

Under normal physiological conditions, ROS are controlled effectively by antioxidants and antioxidases (Stocker, R.; Frei, B. Endogenous antioxidant defenses in human blood plasma. In Oxidative stress, oxidants and antioxidants. H. Sies, editor. London, Academic Press, 1991, 213-243). However, the levels of antioxidants and antioxidases are dramatically depressed in patients suffering from arthritis (Miesel, R.; Zuber, M.; Hartung, R.; Haas, R.; Kroger, H. Total radical-trapping antioxidative capacity of plasma and whole blood chemiluminescence in patients with inflammatory and autoimmune rheumatic diseases. Redox Report, 1995a, 1, 323-330; Miesel, R.; Zuber, M. Copper-independent antioxidase defenses in inflammatory arthritis and autoimmune rheumatic diseases. Inflammation, 1993, 17, 283-294), destabilizing the balance between pro and antioxidant level. Subsequently, the collapse of the antioxidant system causes severe disturbance of the regulatory loop between ROS, antioxidants, antioxidases, NF-κB, and cytokine expression. Four- to five-fold elevated levels of ROS is routinely found in whole blood of mice suffering from collagen-induced arthritis (Miesel, R.; Dietrich, A.; Brandi, B.; Kurpisz, M.; Kroger, H. The phagocytic suppression of proinflammatory response by an active center analogue of Cu₂Zn₂-superoxide dismutase modulates the onset, progression and mission of arthritis. Rheumatol. Int. 1994, 14, 119-126). Up to ten-fold increased ROS was recently shown in patients with inflammatory and autoimmune rheumatic diseases (Miesel, R.; Hartung, R.; Kroger, H. Priming of NADPH oxidase by tumor necrosis factor alpha in patients with inflammatory and autoimmune rheumatic diseases. Inflammation, 1996, 20, 427-438).

Inhibition of ROS production by selective inhibitors of NADPH oxidase ('T Hart, B. A.; Simons, J. M.; Knaan-Shanzer, S.; Bakker, N. P. M.; Labadie, R. P. Antiarthritic activity of the newly developed neutrophil oxidative burst antagonist apocynin. Free Radical Biol. Med. 1990, 9, 127-131; Miesel, R.; Sanocka, D.; Kuprisz, M.; Kroger, H. Anti-inflammatory effects of NADPH oxidase inhibitors. Inflammation, 1995b, 19, 347-362) and a serum stable active center analogue of Cu₂Zn₂ superoxide dismutase (SOD) have been shown to be exceptionally effective in suppressing the development of arthritis in both inflammatory and autoimmune animal models of arthritis (Miesel, R.; Haas, R. Reactivity of an active center analogue of Cu₂Zn₂-superoxide dismutase in a murine model of acute and chronic inflammation. Inflammation, 1993, 17, 595-611; Miesel et al., 1994). The exciting and remarkable clinical success of the recently introduced Enbrel (Immunx Corp), a soluble TNF-α receptor, and Remicade (Johnson and Johnson Corp), a TNF-α-binding antibody, further support the notion that suppression of ROS production and/or counteracting their damaging effects is a valid concept for the successful development of antiarthritic drugs.

Apocynin significantly suppressed the production of TNF-α and IL-1 when added to bacterial antigen-stimulated (mycobacterial 60 kDa heat shock protein, 5 μg/ml) cultures of peripheral blood mononuclear cells (PBMNC) isolated from six patients with RA. At a concentration of 100 μg/ml, apocynin inhibited greater than 50% of the production of TNF-α and IL-1 (Lafeber, F. P. J. G.; Beukelman, C. J.; van den Worm, E.; van Roy, L. L. A. M.; Vianen, M. E.; van Roon, J. A. G.; van Dijk, H.; Bijlsma, J. W. J. Apocynin, a plant-derived, cartilage-saving drug, might be useful in the treatment of rheumatoid arthritis. Rheumatology, 1999, 38, 1088-1093).

In another experiment, apocynin dose-dependently inhibited TNF-α release in both the lipopolysaccharide (LPS)- and staphylococcal peptidoglycan (PG)-stimulated cultures of PBMNC isolated from healthy human donors (Mattsson, E.; van Dijk, H.; van Kessel, K.; Verhoef, J.; Fleer, A.; Rollof, J. Intracellular pathways involved in tumor necrosis factor-α release by human monocytes on stimulation with lipopolysaccharide or staphylococcal peptidoglycan are partly similar. J. Infect. Dis. 1996, 173, 212-218).

In both the phorbol myristate acetate (PMA)- and zymosan-stimulated cultures of neutrophils isolated from the venous blood of healthy human volunteers, apocynin inhibited the ROS production by 50% at a concentration of 4 μg/ml (Simons, J. M.; 'T Hart, B. A.; Ip Vai Ching, T. R. A. M.; van Dijk, H.; Labadie, R. P. Metabolic activation of neutral phenols into selective oxidative burst antagonists by activated human neutrophils. Free Radical Biol. Med. 1990, 8, 251-258). Apocynin also competitively inhibited, in a dose-dependent manner, the production of ROS in PMA-stimulated neutrophils isolated from rats (Salmon, M.; Koto, H.; Lynch, O. T.; Haddad, E.; Lamb, N. J.; Quinlan, G. J.; Barnes, P. J.; Chung, K. F. Proliferation of airway epithelium after ozone exposure, Am. J. Respir. Crit. Care Med. 1998, 157, 970977). The IC₅₀ of apocynin to inhibit NADPH oxidase activity was 9.6 μM.

When peritoneal macrophages isolated from rats were incubated with myelin or zymosan in the presence of fresh normal rat serum (a source of complement), an oxidative burst occurred. Pretreatment of macrophages with apocynin (10 mM) 25 min before the addition of myelin and zymosan significantly reduced the oxidative burst from 75% in controls to about 5% in apocynin-treatment macrophages (van der Goes, A.; Brouwer, J.; Hoekstra, K.; Roos, D.; van den Berg, T. K.; Dijkstra, C. D. Reaction oxygen species are required for the phagocytosis of myelin by macrophages. J. Neuroimmunol. 1998, 92, 67-75).

Apocynin also dose-dependently inhibited the phagocytosis of myelin in these experiments. When J-774A.1 cells, a macrophage-like cell line, are incubated with PMA (50 ng/ml), NADPH oxidase activity was increased from 0.2 to 1.3 nmol superoxide/10⁶ cells. Addition of low density lipoprotein (LDL) to J-774 A.1 cells in the presence of 1 μmol/L CuSO₄ resulted in a time-dependent increase in the release of superoxide to 1.8 nmol superoxide/10⁶ cells (Aviram, M.; Rosenblat, M.; Etzioni, A.; Levy, R. Activation of NADPH oxidase is required for macrophage-mediated oxidation of low-density lipoprotein. Metabolism, 1996, 45, 1069-1079). Addition of apocynin (100 μg/ml) to the incubation system (cells+LDL+Cu²⁺ or cells+PMA) completely blocked the release of superoxide to the medium. While addition of apocynin alone to the cells had no significant effect on the extent of macrophage-released superoxides (0.2 nmol superoxide/10⁶ cells), indicating that apocynin only acts on already activated macrophages.

Type II collagen-Induced arthritis (CIA) is a commonly used rodent model of joint inflammation. Neutrophils play an important role in the pathogenesis of CIA in rats, because depletion of these cells reduces joint inflammation by more than 60%. Furthermore, ROS are implicated in the disease process because SOD reduces disease activity in CIA rats. When male WAG/Rij rats, 10-12 weeks old, were immunized by intracutaneous injection of 1 mg of type II collagen, inflammation of the ankle joints in the hind legs started 12 days later ('T Hart et al., 1990). Apocynin significantly inhibited the joint inflammation. At the lowest dose tested (24 μg/kg), apocynin protected the animals against joint inflammation. Increasing the concentration of apocynin reduced the protective effect, which may be explained by the fact that apocynin at high concentrations blocks its metabolic activation by directly inhibiting the MPO activity (Simons et al., 1990). However, apocynin again inhibited joint swelling at the highest dose tested (200 μg/ml). Apocynin also reduced IL-6 production in these animals. Termination of apocynin treatment did not result in a flare-up of the swelling. This experiment demonstrates that apocynin had good anti-arthritic activity at very low concentrations with an excellent safety profile (apocynin injected to Balb/c mice at a dose of 400 mg/kg had no obvious effects on the mice).

The plant from which apocynin is derived has a long history of safe use for rheumatological diseases (Bensky and Gamble, 1986). The in vitro studies of Lafeber et al., (1999) demonstrate that apocynin can counteract human RA-inflammation-mediated cartilage destruction without having adverse effects on human cartilage. In a phase I human clinical trial in patients with lung emphysema, patients given 12 mg/d (through inhalation) of apocynin for four days showed no side effects (Lafeber et al, 1999).

Stimulated neutrophils release ROS and MPO, which metabolically activate apocynin. The reaction products, which have not been identified with certainty, prevent NADPH assembly by interfering with the intracellular translocation of the two cytosolic components, p47-phox and p67-phox (Stolk, J.; Hilterman, T. J. N.; Dijkman, J. H.; Verhoeven, A. J. Characteristics of the inhibition of NADPH oxidase activation in neutrophils by apocynin, a methoxy-substituted catechol. Am. J. Respir. Cell. Mol. Biol. 1994, 11, 95-102). Thus, cells that lack MPO are generally insensitive to apocynin, indicating that apocynin activation is principally applicable to activated macrophages and neutrophils, which have the capacity to release MPO. For this reason, apocynin will leave the phagocytotic capacity of the neutrophils intact (Thompson, D. K.; Norbeck, L. I.; Olsson, D.; Constantin-Teodosiu, D.; van der Zee, J.; Moldeus, P. Peroxidase-catalyzed oxidation of eugenol: formation of (a) cytotoxic metabolite(s). J. Biol. Chem. 1989, 264, 1016-1021; Simons et al., 1990; Stolk et al., 1994). This demonstrates that apocynin will not compromise the phagocytic system.

However, apocynin does have anti-inflammatory activity because it interferes with arachidonic acid metabolism, and increases the production of prostaglandin E2 by guinea pig pulmonary macrophages (Engles, F.; Renirie, B. F.; 't Hart, B. A.; Labadie, R. P.; Nijkamp, F. P. Effects of apocynin, a drug isolated from roots of Picrorhiza kurroa, on arachidonic acid metabolism. FEBS Lett. 1992, 305, 254-256). Enhanced levels of prostaglandin E2 raises cAMP levels, resulting in the suppression of TNF-α production (Endres, S.; Fulle, H. J.; Sinha, B.; et al., Cyclic nucleotides differentially regulate the synthesis of tumor necrosis factor-α and interleukin-1β by human mononuclear cells. Immunology, 1991, 72, 56-60).

In addition, the mechanism(s) of action of apocynin is (are) clearly different from those of other compounds or antibodies. Thus, apocynin may be effective in RA patients who are not responding well to other drugs.

TNF-α-induced apoptosis in U937 monocytic leukemia cells can be used to evaluate the mechanism of apoptosis and its pharmacological manipulation in various diseases, including inflammation. This can be measured by internucleosomal DNA cleavage (Wright, S. C.; Kumar, P.; Tam, A. W.; Shen, N.; Varma, M.; Larrick, J. W. Apoptosis and DNA fragmentation precede TNF-induced cytolysis in U937 cells. J. Cell. Biochem., 1992, 48, 344-355). It has been shown that signal transduction pathways leading to apoptosis depend on the generation of free radicals (Buttke, T. M.; Sandstrom, P. A. Oxidative stress as a mediator of apoptosis. Immunol. Today, 1994, 15, 7-10) and alterations in the intracellular redox status through depletion of oxidized glutathione (GSH) (Ghibelli, L.; Coppola, S.; Rotilio, G.; Lafavia, E.; Maresca, V.; Ciriolo, M. R. Nonoxidative loss of gluthione in apoptosis via GSH extrusion. Biochem. Biophys. Res. Comm. 1995, 216, 313-320; van den Dobbelsteen, D. J.; Stefen, C.; Nobel, I.; Schlegel, J.; Cotgreave, I. A; Orrenius, S.; Slater, A. R. G. Rapid and specific efflux of reduced glutathione during apoptosis induced by anti-Fas/APO-1 antibody. J. Cell. Biochem., 1996, 271, 15420-15427).

Apocynin has been evaluated in this manner to determine if it can affect the apoptotic pathway. It was discovered that apocynin, and some of the derivatives described herein, generally dose-independently inhibited TNF-α induced DNA-fragmentation in U937 cells (Table 1). Furthermore, the IC₅₀ values suggest that the derivatives 1 and 4 of FIG. 1 are even more potent than apocynin with respect to such bioactivity.

The precise mechanism of inhibition of TNF-α induced DNA-fragmentation in U937 cells by apocynin and derivatives is not known. However, treatments that prevent GSH depletion also protect a cell from apoptosis (Ghibelli, L.; Fanelli, C.; Rotillo, G.; Lafavia, E.; Coppola, S.; Colussi, C.; Civitareale, P.; Ciriolo, M. R. Rescue of cells from apoptosis by inhibition of active GSH extrusion. FASEB, J. 1998, 12, 479-486; Wright, S. C.; Wang, H.; Wei, Q. S.; Kinder, D. H.; Larrick, J. W. Bcl-2-mediated resistance to apoptosis is associated with glutathione-induced inhibition of AP24 activation of nuclear DNA fragmentation. Cancer Res. 1998, 58, 5570-5576), it is believed that this may be a mechanism of action of apocynin. Pretreatment of BALF rats (5 mg/kg) orally with apocynin almost completed inhibited the decrease of glutathione levels induced by ozone exposure (Salmon et al., 1998). Air-exposed rats showed a mean redox ratio of 15.4%. Following ozone exposure, the mean redox value increased to 32.0%, indicating oxidation of glutathione. Apocynin pretreatment reduced the redox value to 18.3%, indicating an antioxidant effect (actual levels of GSH and GSSG were given in the reference, Salmon et al., 1998).

The following Table 1 shows results from and experiment performed according to previously described methods (Wright, S. C.; Zheng, H.; Zhong, J.; Torti, F. M.; Larrick, J. W. Role of protein phosphorylation in TNF-induced apoptosis: phosphatase inhibitors synergize with TNF to activate DNA fragmentation in normal as well as TNF-resistant U937 variants. J. Cell. Biochem., 1993, 53, 222-233).

TABLE 1
Protective effect of apocynin derivatives against
TNF-induced DNA fragmentation in U937 cells*

| Compound | IC₅₀ (μg/ml) |
|---|---|
| apocynin | 64 |
| 1 | 43 |
| 2 | 90 |
| 3 | 80 |
| 4 | 37 |

The foregoing experimental observations related to other anti-inflammatory aspects of apocynin, and the various modified molecules thereof as described herein, demonstrate certain aspects of these compounds' characteristics and bioactivities that are considered highly beneficial for use in treating restenosis. In one regard, inflammation is a substantial culprit in the restenotic cascade. As a result, anti-inflammatory approaches have generally shown promising results, either alone or in combination with other agents, for inhibiting restenosis following angioplasty and/or stenting.

In addition, vulnerable plaque is an area of heightened interest in interventional cardiology. Vulnerable plaques are lesions within the vasculature that have not necessarily progressed to the point of clinical relevance, but exhibit certain qualities that are predisposed toward rupture or otherwise rapid progression toward substantial and threatening occlusions. Such plaques are often characterized as inflamed tissues, and in fact various diagnostic approaches have been investigated to determine the “vulnerability” of certain plaques based on measured parameters indicating levels of inflammation. Once so diagnosed as inflamed and vulnerable, new therapies may be highly beneficial for prophylaxis against the vulnerable progression of the disease state there.

Of particular interest in this setting is anti-inflammatory approaches for prophylaxis and treatment of plaques that are recognized as “vulnerable.” Accordingly, apocynin, and the modified molecules related thereto as described herein, are considered in further embodiments to be highly beneficial agents for treating vulnerable plaques. Such may be locally delivered according to the various embodiments described herein, including without limitation eluting or delivering in conjunction with stents.

The disclosures of these references cited above with respect to this portion of the present description related to apocynin are incorporated herein in their entirety by reference thereto.

RGDfV and Other RGD Peptides

Compounds known as “RGD” peptides are also considered useful embodiments contemplated hereunder for use according to certain of the aspects described herein. One particular highly beneficial embodiment is the compound known as RGDfV, or analogs or derivatives thereof, or pharmaceutically acceptable salts thereof. RGDfV is generally represented by the molecule shown in FIG. 4.

This molecule has been recognized, among other things, as a potent anti-angiogenic factor, intervening via αvβ3 antagonism, and further effecting matrix metalloproteinase (MMP2). Such molecule is considered a beneficial embodiment for use according to various aspects described herein.

Substantial work has been performed in evaluating various biochemical aspects that relate to RGDfV, either directly or indirectly, and its mechanisms and beneficial uses. Further information related thereto is disclosed in one or more of the following references:
Abbruzzese, J. L.; Madden, T.; Newman, R. A. Phase I clinical and pharmacokinetic trial of KW-2189 in patients with solid tumors. Pro. Amer. Asso. Cancer Res. 1996, 37, 165;
Albelda, S. M.; Mette, S. A.; Elder, D. E.; Stewart, R.; Damjanovich, L.; Herlyn, M.; Buck, C. A. Integrin distribution in malignant melanoma: association of the beta3 subunit with tumor progression. Cancer Res. 1990, 50, 6757-6764;
Alberts, S. R.; Erlichman, C.; Erid, J. M.; Ames, M. M.; Sloan, J. A.; Richardson, R. L Pro. Amer. Asso. Cancer. Res. 1996, 37, 393;
Allman, R.; Cowburn, P.; Mason, M. In vitro and in vivo effects of a cyclic peptide with affinity for the αvβ3 integrin in human melanoma cell. Eur. J. Cancer, 2000, 36, 410-422;
Arap, W., Pasqualini, R., Ruoslahti, E. Cancer treatment by targeted drug delivery to tumor vasculature in a mouse model. Science 1998, 279, 377-380;
Aristoff, P. A.; Johnson, P. D. Synthesis of CBI-PDE-I-dimer, the benzannelated analogue of CC-1065. J. Org. Chem. 1992, 57, 6234-6239;
Aristoff, P. A. CC-1065 Analogs: Sequence Specific DNA-alkylating Antitumor Agents. Adv. in Med. Chem. 1993, 2, 67-110;
Aristoff, P. A.; Johnson, P. D.; Daekyu Sun.; Hurley, L. H. Synthes analog of (+)-CC-1065 that also produces delayed toxicity in mice. J. Med. Chem. 1993, 36, 1956-1963;
Auerbach, W., Auerbach, R. Angiogenesis inhibition: a review. Pharmac. Ther. 1994, 63, 265-311;
Barth, A.; Wanek, L. A.; Morton, D. L. Prognostic factors in 1,521 melanoma patients with distant metastases. J. Am. Coll. Surg. 1995, 181, 193-201;
Boehm, T.; Folkman, J.; Browder, T.; O'Reilly, M. S. Nature 1997, 390, 404-407;
Boger, D. L.; Johnson, D. S. CC-1065 and Duocarmycins: Unraveling the keys to a new class of naturally derived DNA alkylating agents. Proc. Natl. Acad. Sci. U.S.A. 1995, 92, 3642-3649;
Bosman, F. T. Integrins: Cell adhesives and modulators of cell function. Histochem. J. 1993, 25, 469-477;
Brooks, P. C.; Montgomery, A. M.; Rosenfeld, M.; Reisfeld, R. A.; Hu, T.; Klier, G.; Cheresh, D. A. Integrin alphavbeta3 antagonists promote tumor regression by inducing apoptosis of angiogenic blood vessels. Cell. 1994, 79, 1157-1164;
Brooks, P. C.; Silletti, S.; von Schalscha, T. L.; Friedlander, M.; Cheresh, D. A. Disruption of angiogenesis by PEX, a noncatalytic metalloproteinase fragment with integrin binding activity. Cell 1998, 92, 391-400;
Brooks, P. C.; Stromblad, S.; Sanders, L. C.; von Schalscha, T. L.; Aimes, R. T.; Stetler-Stevenson, W. G. Localization of matrix metalloproteinase MMP-2 to the surface of invasive cells by interaction with integrin alphavbeta3. Cell, 1996, 85, 683-693;
Brown, J. M.; Siim, B. G. Hypoxia-specific cytotoxins in cancer therapy. Semin. Radiat. Oncol. 1996, 6, 22-36;
Brown, J. M, Lemmon M J. Potentiation by the hypoxic cytotoxin SR4233 of cell killing produced by fractionated irradiation of mouse tumors. Cancer Res. 1990, 50, 7745-7749;
Burris, H. A.; Dieras, V. C.; Tunca, M.; Earhart, R. H.; Eckardt, J. R.; Rodriguez, G. I.; Shaffer, D. S.; Fields, S. M.; Campbell, E.; Schaaf, L.; Kasunic, D.; Von Hoff, D. D. Phase I study with the DNA sequence-specific agent adozelesin. Anti-Cancer Drugs 1997, 8, 588-596;
Burrows, F. Thorpe, P. E. Vascular targeting-a new approach to the therapy of solid tumors, Pharmac. Ther. 1994, 64, 155-174;
Butryn, R. K.; Smith, K. S.; Adams, E. G.; Abraham, I.; Stackpole, J.; Sampson, K. E.; Bhuyan, B. K. Cancer Chemother. Pharmacol. 1994, 34, 44-50;
Byzova, T. V.; Goldman, C. K.; Pampori, N.; Thomas, K. A.; Bett, A.; Shattil, S. J.; Plow, E. F. A mechanism for modulation of cellular responses to VEGF: Activation of the integrins. Mol. Cell, 2000, 6, 851-860;
Carter, C. A.; Waud, W. R.; Li, L. H.; Dekoning T. F.; McGovren, J. P.; Plowman, J. Preclinical antitumor activity of bizelesin in mice. Clinic. Cancer Res., 1996, 2, 1143-11549;
Charo, I. F.; Nannizzi, L'; Smith, J. W.; Cheresh, D. A. The vitronection receptor α5β1 binds fibronectin and acts in concert with αvβ3 in promoting cellular attachment and spreading on fibronectin. J. Cell. Biol., 1991, 111, 2795-2800;
Chiang, S. Y.; Welch, J.; Rauscher III, F. J.; Beerman, T. A. Effects of minor groove binding drugs on the interaction of TATA box binding protein and TFIIA with DNA. Biochemistry 1994, 33, 7033-7040;
Cheresh, D. A. Human endothelial cells synthesize and express an Arg-Gly-Asp directed receptor involved in attachment to fibrinogen and von Willebrand factor. Proc. Natl. Acad. Sci. USA 1987, 84, 6471-6471;
Cheresh, D. A.; Spiro, R. C. Biosynthesis and functional properties of an Arg-Gly-Asp directed receptor involved in human melanoma cell attachment to vitronectin, fibrinogen and von Willebrand factor. J. Biol. Chem. 1987, 262, 17703-17711;
Denekamp, J. Vascular attack as a therapeutic strategy for cancer. Cancer Metast Rev. 1990, 9, 267-282;
D'Incalci, M.; Sessa, C. DNA minor groove binding ligands: a new class of anticancer agents. Expert Opin. Invest. Drugs. 1997, 6, 875-884;
Dorie, M. J.; Brown, J. M. Modification of the antitumor activity of chemotherapeutic drugs by the hypoxic cytotoxic agent tirapazamine. Cancer Chemother. Pharmacol. 1997, 39:361-366;
Erdreich-Epstein, A.; Shimada, H.; Groshen, S.; Liu, M.; Metelitsa, L. S.; Kim, K. S.; Stins, M. F.; Seeger, R. C.; Durden, D. L. Integrins αvβ3 and αvβ5 are expressed by endothelium of high-risk neuroblastoma and their inhibition is associated with increased endogenous ceramide, Cancer Res. 2000, 60, 712-721;
Felding-Habermann, B.; Mueller, B. M.; Romerdahl, C. A. Cheresh, D. A. Involvement of Integrin alpha v gene expression in human melanoma tumorigenicity. J. Clin. Invest 1992, 89, 2018-2022;
Ferrara, N.; Alitalo, K. Clinical applications of angiogenic growth factors and their inhibitors. Nat. Med. 1999, 5, 1359-1364;
Filippini, C.; Bisiach, M.; Tagliabue, G.; D'Incalci, M.; Ubezio, P. Hematopoietic toxicity and cell cycle perturbatiqons induced by new DNA minor groove-alkylating agents. Br. J. Cancer 1997, 72, 801-809;
Fleming, G. F.; Ratain, M. J.; O'Brien, S. M.; Schilsky, R. L.; Hoffman, P. C.; Richards, J. M.; Vogelzang, N. J.; Kasunic, D. A.; Earhart, R. H. Phase I study of adozelesin administration by 24-hour continuous Intravenous infusion. J. Natl. Cancer. Inst. 1994, 86, 368-372;
Folkman, J. Tumor angiogenesis: therapeutic Implications. N. Eng. J. Med. 1971, 285, 1182-1186;
Folkman, J. Tumor angiogenesis, in Mendelson J, Howley P. M., Israel, M. A., et al., (eds): The Molecular Basis of Cancer. Philadelphia, Pa., Saunders, 1995, pp 206-232;
Folkman, J. Addressing tumor blood vessels. Nature Biotech. 1997, 15, 510;
Foster, B. J.; LoRusso, P. M.; Poplin, E.; Zalupski, M.; Valdivieso, M.; Wozniak, A.; Flaherty, L.; Kasunic, D.; Earhart, R. H.; Baker, L. H. Phase I trial of adozelesin using the treatment schedule of daily×5 every 3 weeks. Invest. New Drugs 1996, 13, 321-326;
Friedlander, M.; Brooks, P. C.; Shaffer, R. W.; Kincaid, C. M.; Varner, J. A.; Cheresh, D. A. Definition of two angiogenic pathways by distinct aᵥ integrins. Science 1995, 270, 1500-1502;
Gatenby, R. A.; Kessler, H. B.; Rosenblum, J. S, et al., Oxygen distribution in squamous cell carcinoma metastases and its relationship to outcome of radiation therapy. Int. J. Radiat. Oncol. Biol. Phys. 1998. 14:831-838;
Hammes, H-P., Brownlee, M., Jonczyk, A., Sutter, A., Preissner, K. T. Subcutaneous injection of a cyclic peptide antagonist of vitronectin receptor-type integrins inhibits retinal neovascularization. Nature Med. 1996, 2, 529-533;
Hart, I. R. The selection and characterization of an invasive variant of the B16 melanoma. Am. J. Pathol. 1979, 97, 587-600;
Hieken T. J.; Ronan, S. G.; Farolan, M.; Shilkaitis, A. L.; Das Gupta, T. K. Molecular prognostic markers in intermediate-thickness cutaneous malignant melanoma. Cancer, 1999, 85, 375-382;
Hoffmann, U. B.; Westphal, J. R.; Waas, E. T.; Becker, J. C.; Ruiter, D. J.; van Muijen, G. N. P. Coexpression of Integrin αvβ3 and matrix metalloproteinase-2 (MMP-2) coincides with MMP-2 activation: correlation with melanoma progression. J. Invest Dermatol. 2000, 115, 625-632;
Houghton, P. J.; Cheshire, P. J.; Hallman, J. D. Jr.; Houghton, J. A. Therapeutic efficacy of the cyclopropylpyrroloindole, carzelesin, against xenografts derived from adult and childhood solid tumors. Cancer Chemother. Pharmacol., 1995, 36, 45-52;
Hsu, M. Y.; Shih, D. T.; Meier, F. E.; Van Belle, P.; Hsu, J. Y.; Elder, D. E.; Buck, C. A.; Herlyn, M. Adenoviral gene transfer of beta3 integrin subunit induces conversion from radial to vertical growth phase in primary human melanoma. Amer. J. Pathol. 1998, 153, 1435-1442;
Hynes, R. O. Integrins: versatility, modulation, and signaling in cell adhesion. Cell 1992, 69, 11-25;
Itoh, T.; Tanioka, M.; Yoshida, H.; Yoshika, T.; Nishimoto, H.; Itohara, S. Reduced angiogenesis and tumor progression in gelatinase A-deficient mice. Cancer Res. 1998, 58, 1048-1051;
Kageshita, T.; Hamby, C. V.; Hirai, S.; Kimura, T.; Ono, T.; Ferrone, S. αβv3expression on blood vessel and melanoma cells in primary lesions; differential association with tumor progression and clinical prognosis. Cancer Immunol. Immunother. 2000, 49, 314-318;
Kato, T., Sato, K, Kakinuma, H., Matsuda, Y. Enhanced suppression of tumor growth by combination of angiogenesis inhibitor O-(Chloroacetyl-carbamoyl)fumagillol (TNP-470) and cytotoxic agents in mice. Cancer Res. 1994, 54, 5143-5147;
Keenan, R. M.; Miller, W. H.; Kwon, C.; Ali, F. E.; Callahan, J. F.; Calvo, R. R.; Hwang, S-M.; Kopple, K. D.; Peishoff, C. E.; Samanen, J. M.; Wong, A. S.; Yuan, C-K; Huffman, W. Discovery of potent nonpeptide vitronectin receptor (αvβ3) antagonists. J. Med. Chem. 1997, 40, 2289-2292;
Kobayashi, E.; Okamoto A., Asada, M.; Okabe, M.; Nagamura, S.; Asai, A.; Saito, H.; Gomi, K.; Hirata, T. Characteristics of antitumor activity of KW-2189, a novel water-soluble derivative of duocarmycin, against murine and human tumors. Cancer Res., 1994, 54, 2404-2410;
Koivunen, E., Gay, D. A., Ruoslahti, E. Selection of peptides binding to the α5β1 integrin from phage display library. J. Biol. Chem. 1993, 268, 20205-20210;
Koivunen, E., Wang, B., Ruoslahti, E. Isolation of a highly specific ligand for the α5β1 integrin from a phage display library. J. Cell Biol. 1994, 124, 373-380;
Kraft, S.; Diefenbach, B.; Mehta, R.; Jonczyk, A.; Luckenbach, G. A.; Goodman, S. L. Definition of an unexpected ligand recognition motif for alphavbeta6 integrin. J. Biol. Chem. 1999, 274, 1979-1985;
Kramer, R. H.; Cheng, Y-F.; Clyman, R. Human microvascular endothelial cells using β1 and β3 integrin receptor complexes to attach to laminin. J. Cell. Biol. 1990, 111, 1233-1243;
Kumar, C. C.; Malkowski, M.; Yin, Z.; Tanghetti, E.; Yaremko, B.; Nechuta, T.; Varner, J.; Liu, M.; Smith, E. M.; Neustadt, B.; Presta, M.; Armstrong, L. Inhibition of angiogenesis and tumor growth by SCH221153, a dual αvβ3 and αvβ5 integrin receptor antagonist. Cancer Res. 2001, 61, 2232-2238;
Li, L. H.; Kelly, R. C.; Warpehoski, M. A.; McGovern, J. P.; Gebhard, I.; DeKoning, T. F. Adozelesin, a selected lead among cyclopropylpyrroloondole analogs of the DNA-binding antibiotic CC-1065. Invest. New Drugs 1991, 9, 137-148;
Li, X.; Chen, B.; Blystone, S. D.; McHugh, K. P.; Patrick Ross, F.; Ramos, D. M. Differential expression of av integrins in K1735 melanoma cells. Invasion Metastasis, 1998, 18, 1-14;
Lode, H. N.; Moehler, T.; Xiang, R.; Jonczyk, A.; Gillies, S. D.; Cheresh, D. A.; Reisfeld, R. A. Synergy between an antiangiogeneic integrin αv antagonist and an antibody-cytokine fusion protein eradicates spontaneous tumor metastases;
Lown, J. W.; Wang, Y. and Wei, L. Cyclopropylpyrroloindole-Oligopeptide Anticancer Agents U.S. Pat. No. 5,502,068, 1996;
MacDonald, T. J.; Taga, T.; Shimada, H.; Tabrizi, p.; Zlokovoc, B. V.; Cheresh, D. A.; Laug, W. E. Preferential susceptibility of brain tumors to the antiangiogenic effects of an αv integrin antagonist. Neurosurgery, 2001, 48, 151-157;
McGovern, J. P.; Clarke, G. L.; Pratt, E. A.; DeKoning, T. F. Preliminary toxicity studies with the DNA-binding antibiotic CC-1065. J. Antibiot. 1984, 37, 63-70;
Miller, V. A.; Ng, K. K.; Grant, S. C. et al. Phase II study of the combination of the novel bioreductive agent, tirapazamine, with cisplatin in patients with advanced non-small-cell lung cancer. Ann Oncol. 1997 8:1269-1271;
Mitjans, F.; Meyer, T.; Fittschen, C.; Goodman, S.; Jonczyk, A.; Marshall, J. F.; Reyes, G.; Piulats, J. In vivo therapy of malignant melanoma by means of antagonists of αv integrins. Int. J. Cancer. 2000, 87, 716-723;
Mitjans, F.; Sander, D.; Adan, J.; Sutter, A.; Martinez, J. M.; Jaggle, C. S.; Moyano, J. M.; Kreysch, H. G.; Piulats, J.; Goodman, S. L. An anti-alpha v-integrin antibody that blocks integrin function inhibits the development of a human melanoma in nude mice. J. Cell Sci. 1995, 108, 2825-2838;
Mohler, T., Brooks, P. C., Mitjans, F., Jonczylk, A., Goodman, S., Cheresh, D. A. Antagonists of integrin αvβ3/αvβ5: an anti-angiogenic strategy for the treatment of cancer. Proceeding. Amer. Asso. Cancer Res. 1998, 97;
Montgomery, A. M.; Reisfeld, R. A.; Cheresh, D. A. Integrin αvβ3 rescues melanoma cells from apoptosis in 3D dermal collagen. Proc. Natl. Acad. Sci. USA 1994, 91, 8856-8860;
Moulder, J. E.; Rockwell, S. Tumor hypoxia: its impact on cancer therapy. Cancer Metastases Rev. 1987. 5:313-341;
Nagamura, S.; Kanda, Y.; Kobayashi, E.; Gomi, K.; Saito, H. Synthesis and antitumor activity of duocarmycin derivatives. Chem. Pharm. Bull. 1995, 43, 1530-1535;
Niitani, H.; Horikoshi, N.; Hasegawa, K.; Fukuoka, M.; Kudoh, S.; Hino, M. Phase I study of KW-2189, a derivative of new anticancer antibiotic duocarmycin. Proceeding. Amer. Asso. Cancer Res. 1995, 243;
Nordsmark, M.; Overgaard, M.; Overgaard, J. Pretreatment oxygenation predicts radiation response in advanced squamous cell carcinoma of the head and neck. Radiother. Oncol. 1996. 41:31-40;
Ogasawara, H.; Nishio, K; Takeda, Y.; Ohmori, T.; Kubota, N.; Funayama, Y.; Ohira, T.; Kuraishi, Y.; Isogai. Y.; Saijo, N. A novel antitumor antibiotic, KW-2189 is activated by carboxyl esterase and induces DNA strand breaks in human small cell lung cancer cells. Jpn. J. Cancer Res. 1994, 85, 418-425;
Oku, N., Tokudome, Y., Koike, C., Nishikawa, N., Mon, H., Saiki, I., Okada, S. Liposomal Arg-Gly-Asp analogs effectively inhibit metastatic B16 melanoma colonization in murine lungs. Life Sciences 1996, 58, 2263-2270;
Parker, S. L.; Tong, T.; Bolden, S.; Wingo, P. A. Cancer statistics, 1997, CA Cancer J. Clin. 1997, 47, 5-27;
Petitclerc, E.; Stromblad, S.; Von Schalscha, T. L.; Mitjans, F.; Piulats, J.; Montgomery, A. M.; Cheresh, D. A.; Brooks, P. C. Integrin alpha(v)beta3 promotes M21 melanoma growth in human skin by regulating tumor cell survival. Cancer Res. 1999, 59, 2724-2730;
Reinholt, F. P.; Hultenby, K.; Oldberg, A.; Heinegard, D. Osteopontin—a possible anchor of osteoclasts to bone. Proc. Natl. Aced. Sci. USA, 1990, 87, 4473-4475;
Reynolds, V. L.; Molineux, I. J.; Kaplan, D. J.; Swenson, D. H.; Hurley, L. H. Reaction of antitumor abtibiotic CC-1065 with DNA. Location of the site of thermally induced strand breakage and analysis of DNA sequence specificity. Biochemistry 1985, 24, 6228-6237;
Rockwell, S.; Moulder, J. E. Hypoxic fractions of human tumors xenografted into mice: a review. Int. J Radiat. Oncol. Biol. Phys. 1990. 19:197-202;
Ruoslahti, E. RGD and other recognition sequences for integrins. Annu. Rev. Cell Dev. Biol., 1996, 12:697-715;
Ruoslahti, E., Engvall, E. Perspectives series: cell adhesion in vascular biology. J. Clin. Invest. 1997, 99, 1149-1152;
Sartorelli, A. C. Therapeutic attack on hypoxic cells of solid tumors. Cancer Res. 1988. 48:775-778;
Scatena, M.; Almeida, M.; Chaisson, M. L.; Fausto, N.; Nicosia, R. F.; Giachelli, C. M. NFκB mediates αvβ3 integrin-induced endothelial cell survival. J. Cell. Biol., 1998, 141, 1083-1093;
Siim, B. G.; Menke, D. R.; Dorie, M. J.; Brown, J. M. Tirapazamine-induced cytotoxicity and DNA damage in transplanted tumors: relationship to tumor hypoxia. Cancer Res 1997. 57:2922-2928;
Stromblad, S., Becker, J. C.; Yebra, M.; Brooks, P. C.; Cheresh, D. A. Suppression of p53 activity and p21WAF1/CIP1 expression by vascular cell integrin αvβ3 during angiogenesis. J. Clin. Invest, 1996, 98, 426-433;
Tuszynski, G. P.; Karczewski, J.; Smith, L.; Murphy, A.; Rothman, V. I.; Kundsen, K. A. The GPIIB-IIIa-like complex may function as a human melanoma cell adhesion receptor for thrombospondin. Exp. Cell. Res. 1989, 182, 473-481;
Twentyman, P. R.; Brown, J. M.; Gray, J. W, et al. A new mouse tumor model system (RIF-1) for comparison of end-point studies. J. Natl. Cancer Inst. 1980. 64:595;
Van Hagen, P. M.; Breeman, W. A. P.; Bernard, H. F.; Schaar, M.; Mooij, C. M.; Srinivasan, A.; Schmidt, M. A.; Krenning, E. P.; de Jong, M. Evaluation of a radiolabelled cyclic DTPA-RGD analogue for tumour imaging and radionuclide therapy. Int. J. Cancer (Radiat. Oncol. Invest). 2000, 8, 186-198;
van Tellingen, O.; Punt, C. J. A.; Awada, A.; Wagener, D. J. T.; Piccart, M. J.; Groot, Y.; Schaaf, L. J.; Henrar, R. E. C.; Nooijen, W. J.; Beijnen, J. H. A clinical pharmacokinetics study of carzelesin given by short-term intravenous infusion in a phase I study. Cancer Chemother. Pharmacol. 1998, 41, 377-384;
Wang. Y.; Farquhar, D. Aldophosphamide acetal diacetate and structural analogues: synthesis and cytotoxicity studies. J. Med. Chem., 1991, 34, 197-203;
Wang, Y.; Lown, J. W. Synthesis and structure-activity studies of CC-1065 analogs. Book of Abstracts-209th Amer. Chem. Soc. National Meeting, 1995;
Wang, Y.; Wright, S. C.; Larrick, J. W. DNA-binding indole derivatives, their prodrugs and immunoconjugates as anticancer agents, U.S. Pat. No. 5,843,937, December 1998;
Wang, Y.; Yuan, H.; Ye, W.; Wang, H.; Wright, S. C.; and Larrick, J. W. Synthesis and Preliminary Biological Evaluations of CC-1065 Analogs: Effects of Different Linkers and Terminal Amides on Biological Activity. J. Med. Chem. 2000, 43, 1541-1549;
Wolff, I.; Bench, K; Beijnen, J. S.; Bruntsch, U.; Cavalli, F.; Jong, de. J.; Groot, Y.; Tellingen, O. van; Wanders, J.; Sessa, C. Phase I clinical and pharmacokinetic study of carzelesin (U-80244) given daily for five consecutive days. Clin. Cancer Res. 1996, 2, 1717-1723;
Wright, S. C.; Wei, Q. S.; Zhong, J.; Zheng, H.; Kinder, D. H.; Larrick, J. W. Purification of a 24-kD protease from apoptotic tumors that activated DNA fragmentation. J. Exp. Med. 1994, 180, 2113-2123;
Wright, S. C.; Schellenberger, U.; Wang, H.; Kinder, D. H.; Talhowk, J. W.; Larrick, J. W. Activation of CPP32-like proteases in not sufficient to trigger apoptosis: Inhibition of apoptosis by agents that suppress activation of AP24 but not CPP32-like activity. J. Exp. Med. 1997, 186, 1107-1117;
Wright, S. C.; Schellenberger, U.; Wang, H.; Wang, Y.; Kinder, D. H. Chemotherapeutic drug activation of the AP24 protease in apoptosis: Requirement for caspase 3-like proteases. Biochem. Biophys. Res. Commun. 1998, 245, 797-803; and
Yamaoka, M., Yamamoto, T., Masaki, T., Ikeyama, S., Sudo, K., Fujita, T. Inhibition of tumor growth and metastases of rodent tumors by the angiogenesis inhibitor O-(Chloroacetyl-carbamoyl)fumagillol (TNP-470; AGM-1470). Cancer Res. 1993, 53, 4262-4267.

The disclosures of these references provided in this list immediately above are herein incorporated in their entirety by reference thereto.

Resveratrol and Other Stilbene Compounds

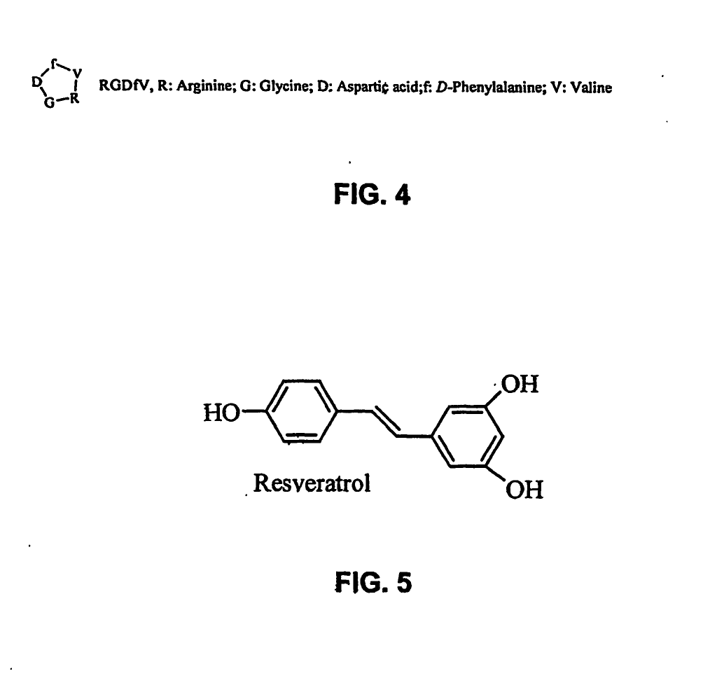

Stilbene compounds are also considered beneficial for use in inhibiting restenosis. One particular beneficial embodiment within this class includes a stilbene compound, Resveratrol, and analogs or derivatives thereof. Resveratrol is generally represented by the molecule shown in FIG. 5.

Further more detailed information regarding this type of compound is variously disclosed In one or more of the following publications:
Baek S J, et al., “Resveratrol enhances the expression of non-steroidal anti-inflammatory drug-activated gene (NAG-1) by increasing the expression of p53,” Carcinogenesis. 2002 March; 23(3): 425-34;
Cheng T H, et al., “Inhibitory effect of resveratrol on angiotensin II-induced cardiomyocyte hypertrophy.” Naunyn Schmiedebergs Arch Pharmacol. 2003 Dec. 9 [Epub ahead of print];
Liu J C, et al., “Inhibition of cyclic strain-induced endothelin-1 gene expression by resveratrol.” Hypertension. 2003 December; 42(6): 1198-205. Epub 2003 Nov. 17;
Lorenz P, et al., “Oxyresveratrol and resveratrol are potent antioxidants and free radical scavengers: effect on nitrosative and oxidative stress derived from microglial cells.” Nitric Oxide. 2003 September; 9(2): 64-76;
Mnjoyan Z H, et al., “Profound negative regulatory effects by resveratrol on vascular smooth muscle cells: a role of p53-p21(WAF1/CIP1) pathway.” Biochem Biophys Res Commun. 2003 Nov. 14; 311(2): 546-52;
Haider U G, et al., “Resveratrol increases serine 15-phosphorylated but transcriptionally impaired p53 and induces a reversible DNA replication block in serum-activated vascular smooth muscle cells.” Mol Pharmacol. 2003 April; 63(4): 925-32;
Haider U G, et al., “Resveratrol suppresses angiotensin II-induced Akt/protein kinase B and p70 S6 kinase phosphorylation and subsequent hypertrophy in rat aortic smooth muscle cells.” Mol Pharmacol. 2002 October; 62(4): 772-7;
Ruef J, et al., “Induction of endothelin-1 expression by oxidative stress in vascular smooth muscle cells.” Cardiovasc Pathol. 2001 November-December; 10(6): 311-5;
Mizutani K, et al., “Phytoestrogens attenuate oxidative DNA damage in vascular smooth muscle cells from stroke-prone spontaneously hypertensive rats.” J Hypertens. 2000 December; 18(12): 1833-40; and
Mizutani K, et al., “Resveratrol inhibits AGEs-induced proliferation and collagen synthesis activity in vascular smooth muscle cells from stroke-prone spontaneously hypertensive rats.” Biochem Biophys Res Commun. 2000 Jul. 21; 274(1): 61-7.

The disclosures of the references in this list are herein incorporated in their entirety by reference thereto.

Camptothecins

The Camptothecin class of compounds includes without limitation, in one beneficial particular embodiment, the DHA-camptothecin class of drug conjugates.

Prior disclosures have indicated the benefits of using such compounds in a beneficial way to treat mammalian cell proliferating disease, e.g., cancer. The present embodiments are in particular related to treating restenosis, and more particularly in relation to in-stent restenosis, utilizing such anti-proliferative properties.

According to certain particular embodiments, conjugates of DHA and camptothecin (CPT) compounds are provided that provide a greatly improved therapeutic efficacy, compared to free camptothecin compounds. These DHA-CPT conjugates have been tested in experimental animal tumor models, and shown excellent antitumor activity compared to the free camptothecin compounds. The DHA-CPT compounds provided according to the present embodiments are used in a beneficial way to treat and/or prevent formation of restenosis.

Further more detailed examples of these compounds are described below according to the following formula (I), or pharmaceutically acceptable salts thereof:
Long chain unsaturated fatty acid-linker-CPT (Formula I)
wherein:

the Long-chain unsaturated fatty acid is generally C₁₂-C₂₂ mono or poly unsaturated fatty acids, which include, but are not limited to, palmitoleic acid, oleic acid, linoleic acid, linolenic acid, arachidonic acid, eicosapentaenoic acid (EPA) and docosahexaenoic acid (DHA);

the linker is -(alkyl)ₘ-(aryl)ₙ-C(O)— or -(aryl)ₘ-(alkyl)ₙ-C(O); wherein: m and n are independently 0-3, and m+n≧1; and

CPT is a camptothecin compound with the following general structure (Formula II): 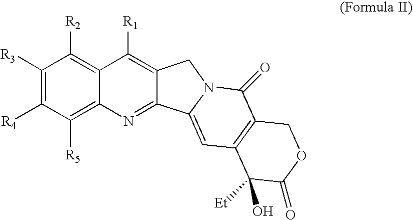
wherein: R₁-R₅ are H, halo, OH, NO₂, NH₂, alkyl, O-alkyl, NH-alkyl, N(alkyl)₂, and can be the same or different. When any of R₁-R₅ is amino, the compounds are the free bases and their acid addition salts, such as HCl and H₂SO₄.

In alternate embodiments of the compounds of formula (I), the fatty acids are DHA and EPA, the linker is selected from Formula III (see below), and CPT, as it is referred to in the present invention, includes the plant alkaloid 20(S)-camptothecin, water insoluble or substantially water insoluble analogs, derivatives, prodrugs and pharmaceutically active metabolites of 20(S)-camptothecin. Examples of camptothecin derivatives include, but are not limited to, 9-nitrocamptothecin, 9-aminocamptothecin, 9-methylcamptothecin, 9-chlorocamptothecin, 9-fluorocamptothecin, 7-ethylcamptothecin, 10-methylcamptothecin, 10-chlorocamptothecin, 10-bromocamptothecin, 10-fluorocamptothecin, 9-methoxycamptothecin, 11-fluorocamptothecin, 10-hydroxycamptothecin, 7-ethyl-10-hydroxycamptothecin, 9-N,N-dimethylaminomethyl-10-hydroxycamptothecin, 10,11-methylenedioxycamptothecin, and 10,11-ethylenedioxycamptothecin, and 7-(4-methylpiperazinomethylene)-10,11-methylenedioxycamptothecin. Prodrugs of camptothecin include, but are not limited to, esterified camptothecin derivatives, such as camptothecin 20-O-propionate, camptothecin 20-O-butyrate, camptothecin 20-O-glycinate, camptothecin 20-O-valerate, camptothecin 20-O-heptanoate, camptothecin 20-O-nonanoate, camptothecin 20-O-crotonate, camptothecin 20-O-2′,3′-epoxybutyrate, nitrocamptothecin 20-O-acetate, nitrocamptothecin 20-O-propionate, and nitrocamptothecin 20-O-butyrate.

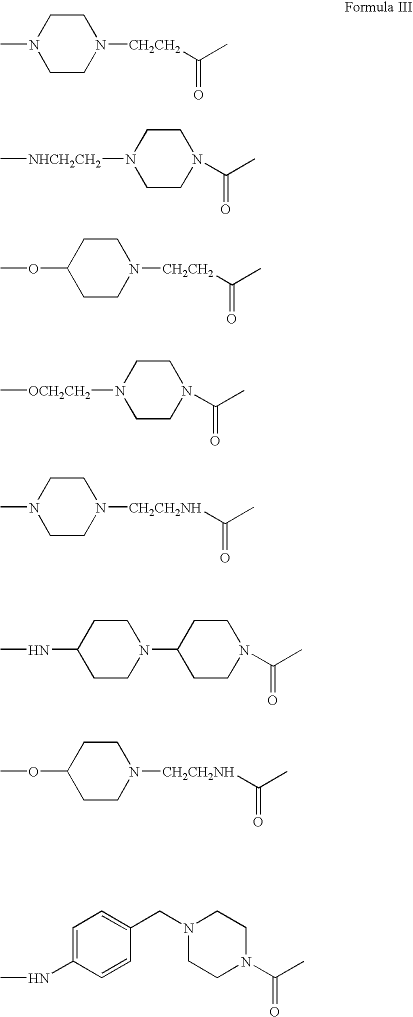

Specific examples of molecules considered beneficial according to various of the embodiments described hereunder are:

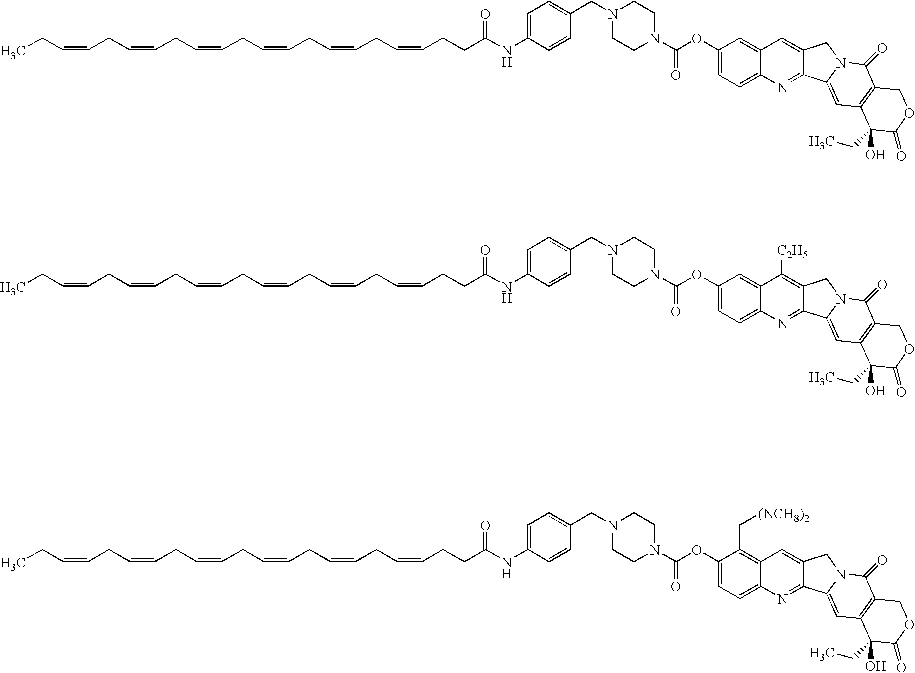

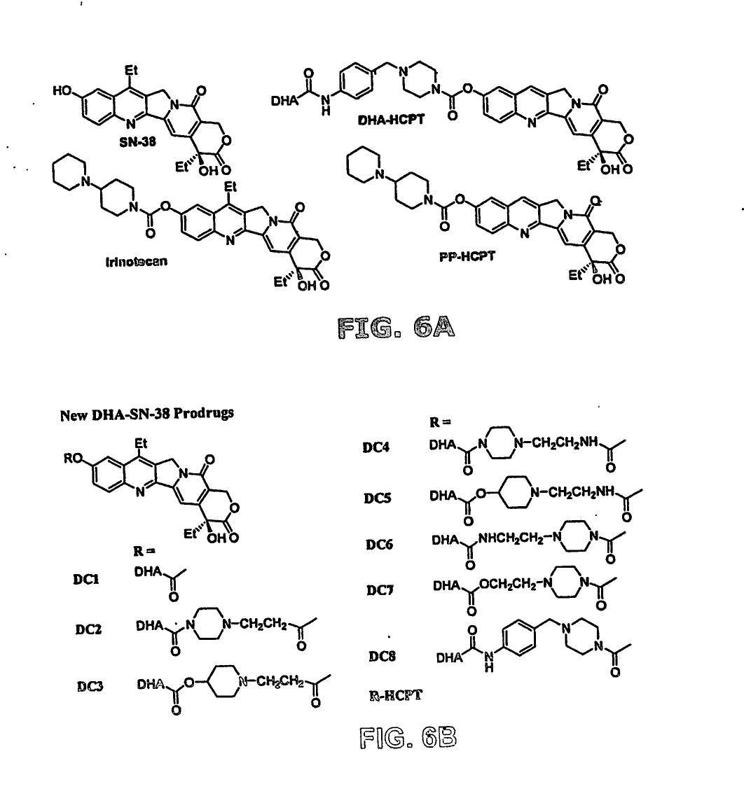

Further related molecules considered beneficial according to various of the embodiments described hereunder are shown in FIGS. 6A-B.

DAA-1 (des-Aspartate-Angiotensin I)

DAA-1 is characterized as an endogenous human short-chain peptide with the following amino acid sequence:

SEQ ID NO:1

Arg-Val-Tyr-IIe-His-pro-Phe-His-Leu.

Further information related to this compound, and in particular relation to its use in treating or preventing restenosis or atherosclerosis, is disclosed in the following issued U.S. Pat. No. 6,100,237 to Sim. The disclosure of this issued U.S. patent is herein incorporated in its entirety by reference thereto.

In addition to the foregoing cited references, further information related to DAA-1 is provided for further understanding as follows.

DAA-I is a naturally occurring biological compound that is endogenous to (i.e., naturally occurring within) human cells, and is a counter-regulatory, cardiovasculo-protective peptide having only a 9 amino acid chain. Prior studies have demonstrated that DAA-I, given both iv and po, has potent protective activity in a number of cardiac and renal pathophysiologies. These activities are believed to be mediated by a novel angiotensin II (ANG-II) receptor subtype that is distinct from the ANG-II site inhibited by ARBs (angiotensin receptor blocker). This activity is further believed to be counter-regulatory to the ANG-II stimulation of VSMC proliferation, and in particular relation to ANG-II stimulated MAP kinase activation (a pathway known to stimulate VSMC proliferation). Further detail of activities conducted with respect to DAA-1 in relation to anti-restenosis applications are provided as follows.

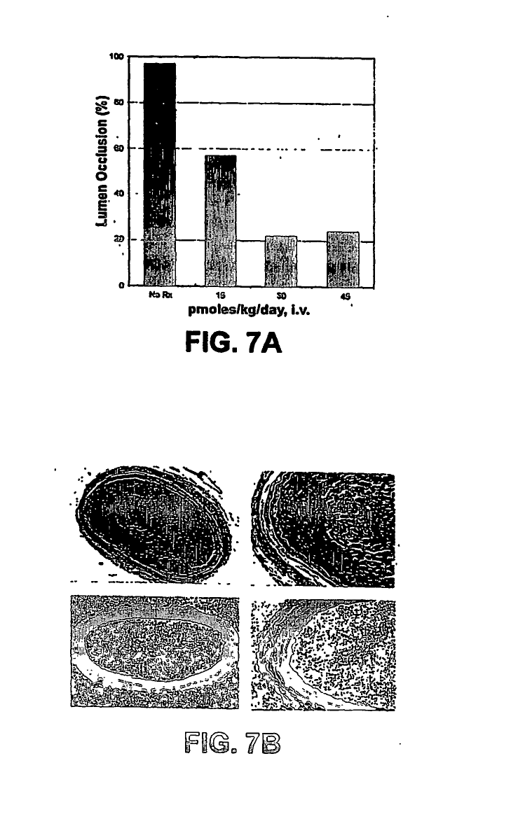

DAA-I has been observed to inhibit balloon-induced intimal injury. Following balloon-induced myocardial intimal injury, rats were give daily intravenous saline or DAA-I at 15, 30 or 45 pmol/kg×14d. FIG. 7A shows a graphical representation of these results compared to control. FIG. 7B shows cross-sectioned histologically prepared slides comparing control sample (shown completely occluded) and representative DAA-1 treated sample. More specifically, the upper slides in FIG. 7B show cross-sectioned results of no-therapy sample, whereas the bottom slides of FIG. 7B show various magnification views of an animal's injured vessel at 14 days following therapy with 30 pmole/kg/day DAA-I. According to these results, DAA-I (in particular at 30-45 pmoles/kg/day iv) provides striking protection from injury-induced lumen restenotic occlusion. Moreover, no appreciable toxicity was detected in the DAA-1 treated animals.

DAA-I is one of two major products of enzymatic conversion of Angiotensin I (“ANG-I”) during normal cellular activity—the other product is Angiotensin II (“ANG-II”). DAA-I is produced when one end of ANG-I, the aspartate end, is enzymatically cleaved by an enzyme. ANG-II results when two amino acids are removed from the C-terminal end of ANG-I. ANG-II is known to bind certain cell surface receptors, resulting in the production of “secondary messengers” that promote cell division and proliferation. A natural balance is believed to exist between DAA-I and ANG-II in normal, healthy, quiescent cells. ANG-II mediated cellular proliferation activities are known to increase within smooth muscle cells of vessel walls post-recanalization injury, and ANG-II is thus considered an active contributor to the biochemical cascade of restenosis. While the specific inter-relationship of DAA-I activity and the ANG-II cascade has not been investigated in detail in the context of smooth muscle cell proliferation, it is believed that the known proliferative activities of ANG-II will be antagonized by elevating the DAA-I levels in the SMCs of injured vessel walls.

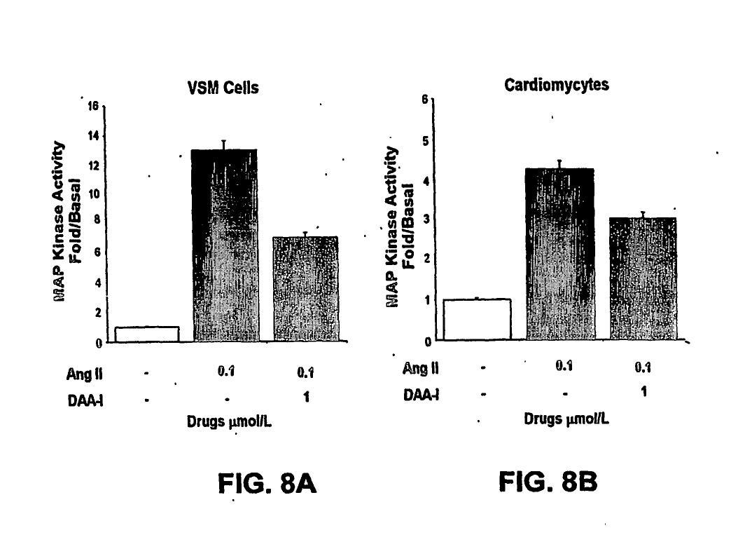

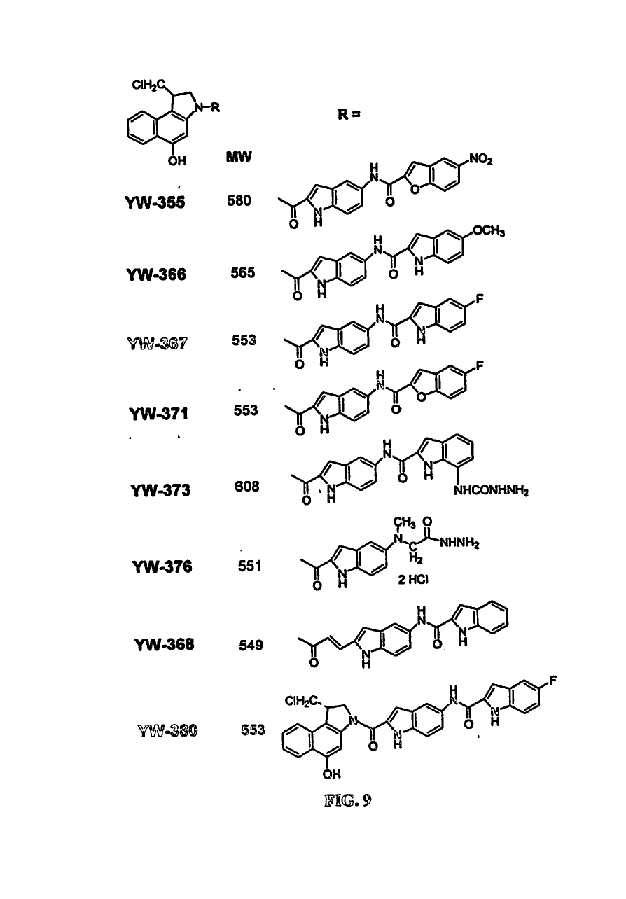

In addition, at least one published study has been performed that indicate DAA-I blocks ANG-II-stimulated MAP kinase production in smooth muscle cells. MAP kinase is believed to promote cell transition early in the cell cycle between G0 to G1 phases, and MAP kinase inhibition has been correlated with reduced smooth muscle cell proliferation. FIGS. 8A-B show a graphical illustration of certain results of one study performed comparing MAP Kinase activity without ANG-II stimulation, with ANG-II stimulation, and with ANG-II stimulation in the presence of DAA-1. More specifically, FIG. 8A shows such results for vascular smooth muscle cells, whereas FIG. 8B shows the results for cardiomyocytes for further illustration. As these results indicate, DAA-1 substantially reduced the ANG-II stimulated MAP Kinase activity in these types of cells.

In addition, other studies have indicated that DAA-I at certain levels attenuates the expression of intercellular adhesion molecule one (ICAM-1), and reduces release of myeloperoxidase (MPO) and serum creatine kinase (CK) post myocardial infarction.

Accordingly, the delivery of DAA-I to injured vessels has been shown to substantially inhibit smooth muscle cell proliferation and drastically reduce restenosis. This is believed to be associated with counter-regulation of Angiotensin II and MAP kinase activities normally found in SMCs of injured arteries. This demonstrated bioactivity has furthermore been shown to be highly potent at mere micro-molar concentrations, as well as safe as an endogenous human peptide being used in the present embodiments in a man-enhanced mode of its suspected role in nature.

“ADF” (Apoptosis DNA Factor)

ADF is the fragment of mitochondrial maleate dehydrogenase (MDH) with the following amino acid sequence:

SEQ ID NO:2

KAKAGAGSATLSMAYAGARFVFSLVDAMNGKEGVVECSFVKSQETECTYF
STPLLLGKKGIEKNLGIGKVSSFEEKMISDAIPELKASIKKGEDFVKTL
K.

This compound, and various appropriate analogs or derivatives thereof, are considered a further embodiment for beneficial use in treating or preventing stenosis or restenosis, and otherwise for use in conjunction with endolumenal stenting.

Further included are certain derivatives or analogs of these compounds. For example, also contemplated is use of the fragment of ADF with the following amino acid sequence:

SEQ ID NO:3

KAKAGAGSATLSMAYAGARFVFSLVDAMNGKEGVVECSFVKSQETECTYF
STPLLLGKKGIEKNLGIGKVSS.

In another regard, homologs of these compounds are also contemplated. In one particular example without limitation, an ADF homolog represented by the substitution of various amino acids giving homologous proteins mediating substantially all of its activity, at least relative to the desired indications described herein.

CC-1065 and Duocarmycin Derivatives

Certain beneficial embodiments incorporate one or more minor groove binders, such as duocarmycin compounds, in the systems and related methods disclosed elsewhere herein for providing local medical therapy to tissues. In one highly beneficial embodiment, a compound known as CC-1065 is used in these assemblies and systems, and for the various purposes described herein. Further information related to this compound and related characteristics and bioactivity is disclosed in the following U.S. Pat. No. 5,843,937 to Wang et al. Further information is disclosed in the following publication: Wang, Y.; Yuan, H.; Ye, W.; Wang, H.; Wright, S. C.; and Larrick, J. W. Synthesis and Preliminary Biological Evaluations of CC-1065 Analogs: Effects of Different Linkers and Terminal Amides on Biological Activity. J. Med. Chem. 2000, 43, 1541-1549. The disclosures of these patent and publication references are herein incorporated in their entirety by reference thereto.

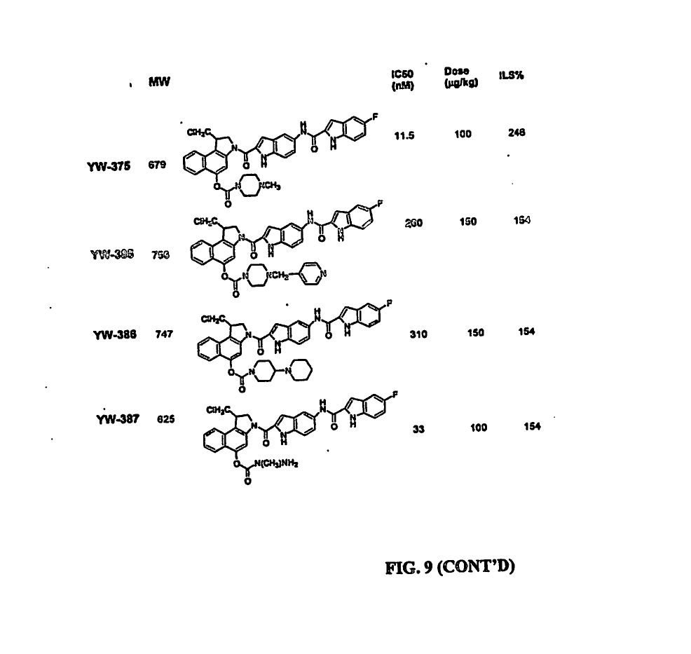

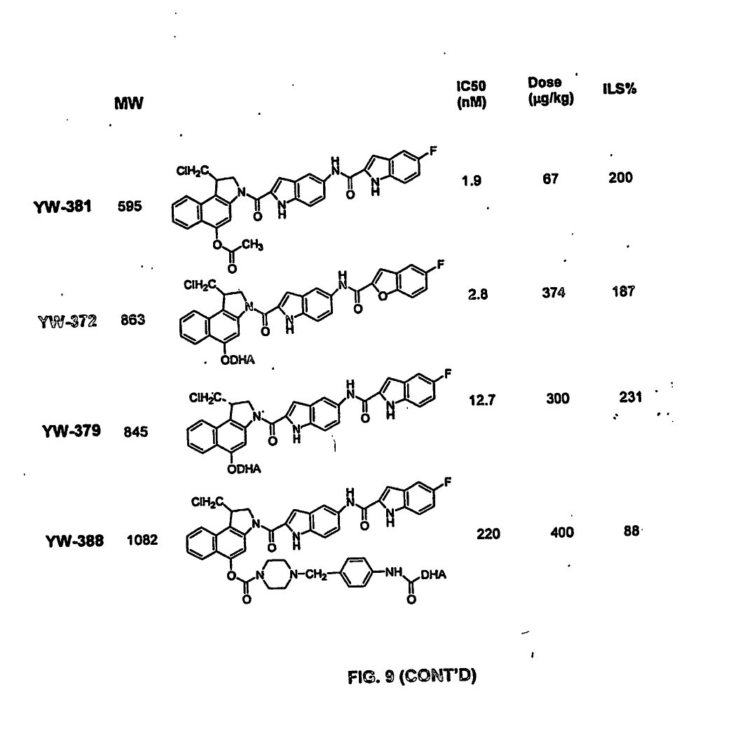

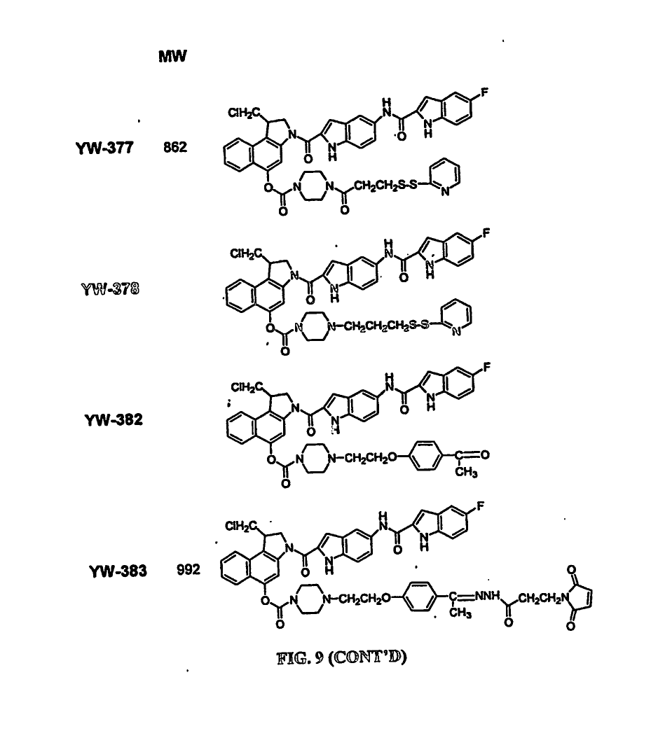

Various modified molecules herein contemplated are shown in FIG. 9. CC-1065 is shown below.

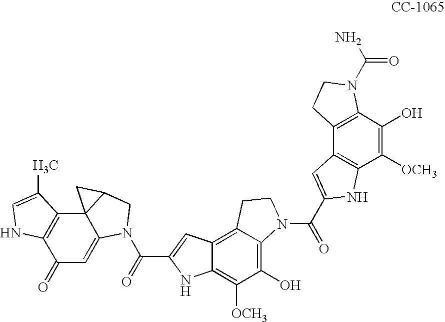

According to the present embodiments, one or more such compounds are used for therapy or prophylaxis of certain medical conditions, generally related to endolumenal stenting or otherwise according to the systems and methods described herein. As described elsewhere herein with respect to this or other compound embodiments, such may be accomplished via stent elution, such as from coatings associated with the stent, or other local delivery or even systemic or oral delivery modalities.

Systems and Methods Incorporating Molecular Embodiments

Various of the molecular embodiments described herein are considered in particular highly beneficial for treating, preventing, or inhibiting endolumenal stenosis, or restenosis such as following a luminal wall injury. In addition, various of these molecular embodiments are further considered useful for use in conjunction with medical device implants such as stents. Such may be for example in order to inhibit restenosis following the stent implant or other injury associated therewith.

However, other beneficial uses, and related systems and methods, are also contemplated. For example, certain of the compounds have demonstrated or been observed to possess certain potent anti-cancer or other anti-inflammatory activities and benefits, and thus may be delivered locally to tissues associated with a stented region in order to achieve such benefits. In one more particular example for illustration, a stent may be implanted for example within a lumen such as a vessel feeding or adjacent to a cancerous tumor or inflamed tissue. Various modes of local delivery of such compounds to that tissue, such as via elution from the stent or as otherwise described herein, are considered further embodiments hereunder.

It is to be appreciated that, among the various molecular approaches to treat medical conditions as described herein, one or more of the agents are believed to provide a certain degree of benefit using oral or otherwise systemic delivery and dosing. Such may be accomplished for example using conventional carrier vehicles (such as for example in pill or liquid form), or other IV or injectable or oral preparations. However, even where certain such molecules might not demonstrate acceptable efficacy and/or safety in such modalities, such may be the result of the systemic application, e.g., dosing and delivery modality of the particular compounds, and not relate to the molecule itself if delivered to the tissue to be treated in another manner. For example, systemic (e.g., IV) or oral dosing of such compounds may be subject to certain clearance, metabolism, or simple dilution aspects that render the treatment compounds ineffective under the particular delivery modality.

Accordingly, certain aspects of the present invention incorporate such compounds in local delivery modalities to maximize the local potency and bioactivity at the site to be treated. For example, such would be local delivery to the site of vascular injury related to restenosis, or in the setting of treating atherosclerosis (including for example as prophylaxis of vulnerable plaque). In general, such terms of “local delivery” in this context, or terms of similar import, are herein intended to mean delivery in a manner that increases the local amount, concentration, or effect of the delivered compound in a biologically relevant manner as compared to systemic delivery, again such as via systemic IV or intramuscular injections etc.

More specifically, local dosing such as through needle injection catheters, or local end-hole or side-hole injection catheters, may provide necessary local concentrations to accomplish the objective of substantial reduction in atherosclerosis in one regard, or restenosis in another regard (or prophylaxis or therapy of vulnerable plaque in another regard). Of particular benefit, incorporating such compounds into or onto drug eluting stents for local elution directly into the subject endolumenal wall is considered a highly beneficial embodiment. In further embodiments, systemic dosing of such compounds is accomplished via complexing the particular molecules with “pro-drug” technologies, which deliver and provide the desired bioactivity only in local target cells such as injured vessel wall lining.

In still a further regard, the specific compounds described herein may be used in combination with other bioactive agents, either in combined form in the respective carrier or delivery mechanism, or in coordination with separate delivery modes (e.g., one as a stent elution coating, the other locally or systemically injected; etc.). For example, the various embodiments may be combined with delivery of other drugs for combined desired effect.

Such combination is provided in a manner to provide for beneficial synergistic results providing therapies with safer and/or more efficacious results. In one particular regard for example, such anti-proliferative compounds delivered at doses that might otherwise have certain local toxicities in the area, e.g., sirolimus or paclitaxel, may gain for example substantial benefit by the combination therapy with one or more of the agents described herein.

Generally well accepted studies and protocols have been published and are well know to characterize and optimize such benefits from particular combinations, or with respect to a specific delivery mode of one of the compounds.

Whereas the present embodiments are considered of particular benefit for treating vascular restenosis, such as in the coronary or peripheral arteries, other vessels or lumens than blood vessels are contemplated as indicated regions of the body where therapeutic uses may be provided. Examples include the binary duct, pancreatic duct, urethra, fallopian tubes, etc., to the extent the intended applications of stent elution, and/or restenosis or stenosis therapy or prevention are related to such areas.

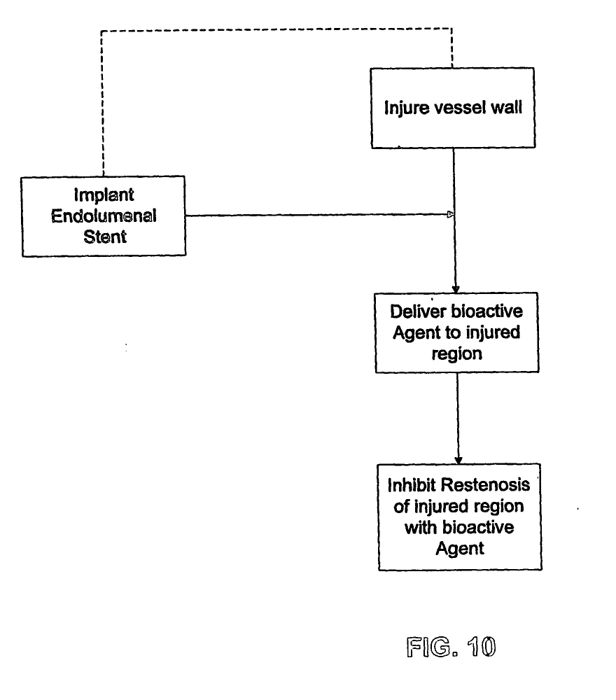

FIG. 10 shows a flow diagram of one embodiment of the invention for delivering one or more of the compounds described herein, or analogs or derivatives thereof, to an injured region of a blood vessel in order to inhibit restenosis. This may be done in conjunction with stenting, shown in dashed line, which stenting may be the procedure by which the injury is made or adjunctive thereto, e.g., after atherectomy or predilation via angioplasty (as shown in alternative arrowed dashed lines).

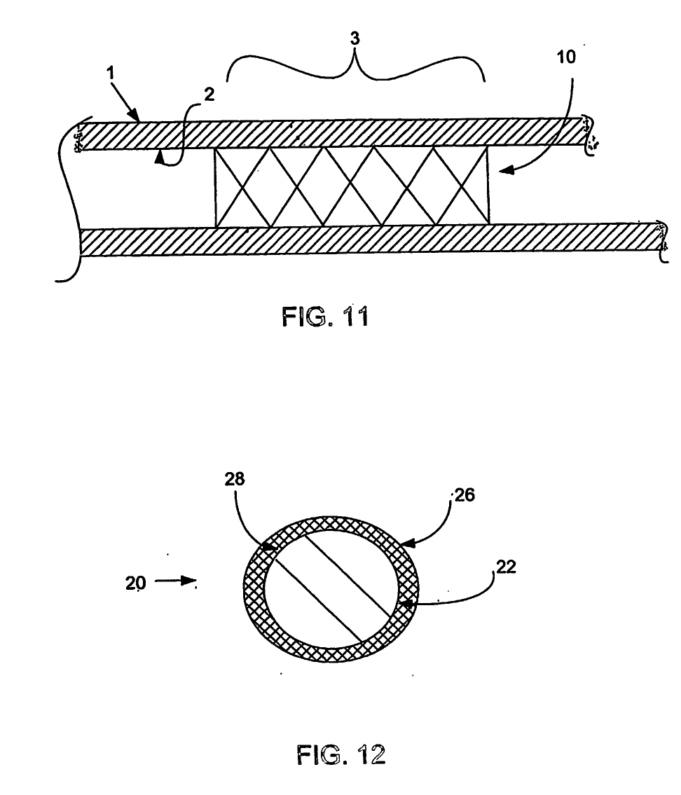

FIG. 11 shows a schematic representation of an artery 1 which is stented with a stent 10 along a stented region 3. The endolumenal vessel lining 2 is typically denuded along the stented region 3. The stent 10 is preferably endothelialized, and the vessel lining 2 is preferably re-endothelialized, while importantly smooth muscle cell hyperproliferation is inhibited, according to the local delivery of the compounds as described herein.

In a highly beneficial mode shown in cross-section in FIG. 12, the bioactive compound or agent 28 is incorporated onto the stent 10 in a coating 26 located over underlying stent strut 22. In any event, incorporation of the particular compounds described herein into or with such devices and compositions are contemplated as highly beneficial embodiments of the present invention. It is also to be appreciated that local delivery of one or more of the compounds described herein, with or in conjunction with such stents, or otherwise to treat or prevent atherosclerosis, stenosis, restenosis, smooth muscle cell proliferation, occlusive disease, or other abnormal lumenal cellular proliferation condition, constitutes, in the various forms apparent to one of ordinary skill, broad aspects of the invention that are not intended to be limited in all cases to the more particular embodiments, though such are independently valuable as would be apparent to one of ordinary skill.

Measurement of the Efficacy of Compounds

The compounds of the present invention function as inhibitors of stenosis and restenosis. The synthesis, selection, and use of the compounds of the present invention, which are capable of modulating stenosis and restenosis is within the ability of a person of ordinary skill in the art. For example, well-known in vitro or in vivo assays can be used to determine the efficacy of various candidate compounds to promote molecular events that modulate smooth muscle cell activation, see, e.g., Lester et al., Endocrine Rev. 10: 420-36 (1989). Further, any in vitro or in vivo assays developed to measure the activity, modification or expression of the molecular markers of cellular activation and proliferation of smooth muscles cells (e.g., cyclin E, cdk2, cyclin A, cyclin D1, and cdk4/6), inflammation activity, or intimal injury may be employed to assess the biological activity (namely, the agonist or antagonist properties) of compounds of the present invention. Several examples of these assays have been described above.

Pharmaceutical Compositions and Formulations

The compounds of the invention, and derivatives, fragments, analogs and homologs thereof, can be incorporated into pharmaceutical compositions suitable for administration. Such compositions typically comprise the nucleic acid molecule, polypeptide, or antibody and a pharmaceutically acceptable carrier. As used herein, “pharmaceutically acceptable carrier” is intended to include any and all solvents, dispersion media, coatings, antibacterial and antifungal compounds, isotonic and absorption delaying compounds, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, Ringer's solutions, dextrose solution, and 5% human serum albumin. Liposomes and non-aqueous vehicles such as fixed oils may also be used. The use of such media and compounds for pharmaceutically active substances is well known in the art. Except insofar as any conventional media or compound is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid (EDTA); buffers such as acetates, citrates or phosphates, and compounds for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal compounds, for example, parabens, chlorobutanol, phenol, ascorbic acid, thimerosal, and the like. In many cases, it will be preferable to include isotonic compounds, for example, sugars, polyalcohols such as manitol, sorbitol, sodium chloride in the composition. Prolonged absorption of the injectable compositions can be brought about by including in the composition a compound which delays absorption, for example, aluminum monostearate and gelatin.

Sterile injectable solutions can be prepared by incorporating the active compound (e.g., a compound or anti-compound antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding compounds, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating compound such as alginic acid, Primogel, or corn starch; a lubricant such as magnesium stearate or Sterotes; a glidant such as colloidal silicon dioxide; a sweetening compound such as sucrose or saccharin; or a flavoring compound such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared as pharmaceutical compositions in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Uses of the Compositions of the Invention as Coatings for Devices

The present invention also provides stents and catheters, comprising a generally tubular structure (which includes for example, spiral shapes), the surface of which is coated with a composition described above. A stent is a scaffolding, usually cylindrical in shape, that may be Inserted into a body passageway (e.g., bile ducts) or a portion of a body passageway, which has been narrowed, irregularly contoured, obstructed, or occluded by a disease process (e.g., ingrowth by a tumor) in order to prevent closure or reclosure of the passageway. Stents act by physically holding open the walls of the body passage into which they are inserted.

Commercially available poly(ethylene oxide) [PEO] and poly (acrylic acid) [PAA] gel-coated balloon angioplasty catheters can be used investigated for their use as local drug delivery systems in terms of get/solute interactions, solute loading, and release kinetics (Gehrke et al., in Intelligent Materials & Novel Concepts for Controlled Release Technologies, S. Dinh and J. DeNuzzio, Eds., ACS Symposium Series, Washington, D.C., 728, 43-53 (1999)). Loading of proteins in PEO-gel coatings can be approximately doubled with the addition of soluble dextran to the loading solution. Release of solutes from gel coatings is diffusion limited, though resistance may be due to the boundary layer as well as the gel.

A variety of stents and catheters may be utilized within the context of the present invention, including, for example, esophageal stents, vascular stents, binary stents, pancreatic stents, ureteric and urethral stents, lacrimal stents, Eustachiana tube stents, fallopian tube stents and tracheal/bronchial stents, vascular catheters, and urethral catheters.

Stents and catheters may be readily obtained from commercial sources, or constructed in accordance with well-known techniques. Representative examples of stents include those described in U.S. Pat. No. 4,768,523, entitled “Hydrogel Adhesive,” U.S. Pat. No. 4,776,337, entitled “Expandable Intraluminal Graft, and Method and Apparatus for Implanting and Expandable Intraluminal Graft;” U.S. Pat. No. 5,041,126 entitled “Endovascular Stent and Delivery System;” U.S. Pat. No. 5,052,998 entitled “Indwelling Stent and Method of Use,” U.S. Pat. No. 5,064,435 entitled “Self-Expanding Prosthesis Having Stable Axial Length;” U.S. Pat. No. 5,089,606, entitled “Water-=insoluble Polysaccharide Hydrogel Foam for Medical Applications;” U.S. Pat. No. 5,147,370, entitled “Nitinol Stent for Hollow Body Conduits;” U.S. Pat. No. 5,176,626, entitled “Indwelling Stent;” U.S. Pat. No. 5,213,580, entitled “Biodegradable polymeric Endoluminal Sealing Process.”

Stents and catheters may be coated with a composition of the invention in a variety of manners, including for example: (a) by directly affixing to the device the composition (e.g., by either spraying the stent with a polymer/drug film, or by dipping the stent into a polymer/drug solution), (b) by coating the device with a substance such as a hydrogel which will in turn absorb the composition, (c) by interweaving the composition coated thread (or the polymer itself formed into a thread) into the device structure, (d) by inserting the device into a sleeve or mesh which is comprised of or coated with the composition, or (e) constructing the device itself with the composition. Within preferred embodiments of the invention, the composition should firmly adhere to the device during storage and at the time of insertion The composition should also preferably not degrade during storage, prior to insertion, or when warmed to body temperature after expansion inside the body. In addition, it should preferably coat the device smoothly and evenly, with a uniform distribution of the composition, while not changing the device contour. Within preferred embodiments of the invention, the release of the composition should be uniform, predictable, and may be prolonged into the tissue surrounding the device once it has been deployed. For vascular stents and catheters, in addition to the above properties, the composition should not render the stent or catheter thrombogenic (causing blood clots to form), or cause significant turbulence in blood flow (more than the stent itself would be expected to cause if it was uncoated).

Patches may also be prepared from materials that contain a composition of the invention. For example, patch materials, e.g., but not limited to, Gelfoam or Polyvinyl alcohol (PVA), or other suitable material, may be used. Such patches may be used prophylactically or therapeutically to deliver the composition when contacted with a cell.

Treatment of Disease and Disorders

A. Prophylactic and Therapeutic Uses of the Compositions of the Invention

The compounds of the present invention are useful in potential prophylactic and therapeutic applications implicated in a variety of disorders in a subject (See Diseases and Disorders). Diseases and disorders that are characterized by increased (relative to a subject not suffering from the disease or disorder) levels or biological activity of smooth muscle cell activation and proliferation can be treated with therapeutic compounds that antagonize (i.e., reduce or inhibit) activity, which can be administered in a therapeutic or prophylactic manner. Increased or decreased levels can be readily detected by obtaining a patient tissue sample (e.g., from biopsy tissue) and assaying it in vitro for levels or biological activity of smooth muscle cell activation. Therapeutic compounds that can be utilized include, but are not limited to: (i) an aforementioned compound, or analogs, derivatives, fragments or homologs thereof; (ii) anti-compound antibodies to an aforementioned compound of the present invention; (iii) polynucleotide encoding an aforementioned compound; or (iv) modulators (i.e., inhibitors, agonists and antagonists, including additional peptide mimetic of the invention or antibodies specific to a peptide of the invention) that alter the interaction between an aforementioned compound and its binding partner.

i. Prophylactic Methods

In one aspect, the invention provides a method for preventing a disease or condition associated with smooth muscle cell activation and proliferation in a subject, by administering to the subject a compound of the invention, a polynucleotide encoding said compound, or a compound mimetic that inhibits smooth muscle cell activation and cellular proliferation.

Subjects at risk for a disease that is caused or contributed to by aberrant smooth muscle cell activation and proliferation can be identified by, for example, any or a combination of diagnostic or prognostic assays as described herein. Administration of a prophylactic compound can occur prior to the manifestation of symptoms characteristic of the aberrancy, such that a disease or disorder is prevented or, alternatively, delayed in its progression. Depending upon the type of aberrancy, for example, a compound, a compound mimetic, or anti-compound antibody, which acts as an antagonist to smooth muscle cell activation and proliferation, the appropriate compound can be determined based on screening assays described herein.

i. Therapeutic Methods

Another aspect of the invention includes methods of inhibiting smooth muscle cell activation and proliferation in a subject for therapeutic purposes. The modulatory method of the invention involves contacting a cell with a compound of the present invention, that inhibits smooth muscle cell activation and cell proliferation. Compounds that inhibits smooth muscle cell activation and proliferation are described herein. These methods can be performed in vitro (e.g., by culturing the cell with the compound) or, alternatively, in vivo (e.g., by administering the compound to a subject). As such, the invention provides methods of treating an individual afflicted with a disease or disorder manifested by aberrant activation of smooth muscle and proliferation. The method can involve administering one compound (e.g., a compound identified by a screening assay described herein), or combination of compounds that inhibit smooth muscle cell proliferation and proliferation.

B. Determination of the Biological Effect of the Therapeutic

Suitable in vitro or in vivo assays are performed to determine the effect of a specific therapeutic and whether its administration is indicated for treatment of the affected tissue in a subject. In vitro assays can be performed with representative cells of the type(s) involved in the patient's disorder, to determine if a given therapeutic exerts the desired effect upon the cell type(s). Compounds for use in therapy can be tested in suitable animal model systems including, but not limited to rats, mice, chicken, cows, monkeys, rabbits, and the like, prior to testing in human subjects. Similarly, for in vivo testing, any of the animal model system known in the art can be used prior to administration to human subjects.

C. Diseases and Disorders

Smooth muscle cell proliferation is associated with numerous diseases, all of which could be effected by the development of a smooth muscle cell proliferation-modulating agent. The invention provides for both prophylactic and therapeutic methods of treating a subject at risk of (or susceptible to) a disorder or having a disorder associated with aberrant smooth muscle cell activation, e.g., but not limited to, uterine fibroid tumors, prostatic hypertrophy, bronchial asthma, portal hypertension in cirrhosis, bladder disease, pulmonary and systemic arterial hypertension, atherosclerosis, and vascular restenosis after angioplasty are thought to be the result of smooth muscle cell activation and excessive smooth muscle cell proliferation.

The disclosures of all the published literature and issued or published patent references provided throughout this disclosure are herein incorporated in their entirety by reference thereto.

Certain particular compounds have been described herein in various assemblies or methods of use as highly beneficial aspects of the invention. However, other analogs or derivatives thereof may be used and contemplated within the intended scope of various aspects of the invention. For example, similar bioactivity as is known for the compounds described may be achieved with modifications to the specific molecule without departing from the intended scope of such aspects. In one regard, active sites and molecular regions or shapes, etc., associated therewith may be incorporated onto other molecular chains and provide further aspects of the invention. Moreover, conjugates or pro-drugs of these compounds are further contemplated, as are the various modes of combination use with each other, or with other therapeutic agents for this indication, as would be apparent to one of ordinary skill upon review of this disclosure in combination with other available art. In a further example, pharmaceutically acceptable salts of the noted compounds are contemplated. Still further, such compounds or their modifications may be incorporated into certain pharmaceutically acceptable carriers as would be apparent to one of ordinary skill.

The various compounds described herein are generally available for purchase, or may be otherwise manufactured or otherwise produced or prepared, using various known methods. Such for example may include purchasing or producing such agents in substantially purified form, or in combination with other agents or additives or byproducts of manufacture, which may be later purified or used in such combination form according to the embodiments described herein. Moreover, the agents described may be packaged together with the respective local delivery modality or adjunctive therapeutic and/or diagnostic devices in overall pre-packaged assemblies. Or, such may be packaged separately for later combination in providing medical therapy, as would be apparent to one of ordinary skill.

Although the description above contains many specificities, these should not be construed as limiting the scope of the invention but as merely providing illustrations of some of the presently preferred embodiments of this invention. Thus the scope of this invention should be determined by the appended claims and their legal equivalents. Therefore, it will be appreciated that the scope of the present invention fully encompasses other embodiments which may become obvious to those skilled in the art, and that the scope of the present invention is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

What is claimed is:

1. A system for treating or preventing atherosclerosis, stenosis, restenosis, smooth muscle cell proliferation, occlusive disease, or other abnormal lumenal cellular proliferation condition providing interventional medical care to a patient, comprising:
a local delivery system;
a bioactive agent;
wherein the local delivery system is adapted to locally deliver the bioactive agent to a region of tissue associated with the condition;
wherein the bioactive agent when locally delivered to the region of tissue is adapted to treat or prevent the condition; and
wherein the bioactive agent comprises at least one of CC-1065, duocarmycin, apocynin, RGDfV, RGD peptide, resveratrol, a stilbene compound, camptothecin, des-aspartate angiotensin I (“DAA-1”), or apoptosis DNA factor (“ADF”), or an analog or derivative thereof, or a pharmaceutically acceptable salt thereof, or a combination or blend thereof.

2. The system of claim 1, wherein the bioactive agent comprises CC-1065 or an analog or derivative thereof, or pharmaceutically acceptable salt thereof.

3. The system of claim 1, wherein the bioactive agent comprises duocarmycin or an analog or derivative thereof, or pharmaceutically acceptable salt thereof.

4. The system of claim 1, wherein the bioactive agent comprises apocynin or an analog or derivative thereof, or a pharmaceutically acceptable salt thereof.

5. The system of claim 1, wherein the bioactive agent comprises RGDfV or an analog or derivative thereof, or a pharmaceutically acceptable salt thereof.

6. The system of claim 1, wherein the bioactive agent comprises an RGD peptide or an analog or derivative thereof, or a pharmaceutically acceptable salt thereof.

7. The system of claim 1, wherein the bioactive agent comprises resveratrol or an analog or derivative thereof, or a pharmaceutically acceptable salt thereof.

8. The system of claim 1, wherein the bioactive agent comprises a stilbene compound or an analog or derivative thereof, or a pharmaceutically acceptable salt thereof.

9. The system of claim 1, wherein the bioactive agent comprises camptothecin or an analog or derivative thereof, or a pharmaceutically acceptable salt thereof.

10. The system of claim 1, wherein the bioactive agent comprises DAA-1 or an analog or derivative thereof, or a pharmaceutically acceptable salt thereof.

11. The system of claim 1, wherein the bioactive agent comprises ADF or an analog or derivative thereof, or a pharmaceutically acceptable salt thereof.

12. The system of claim 1, wherein the bioactive agent comprises the following molecule, or an analog or derivative thereof, or a pharmaceutically acceptable salt thereof:

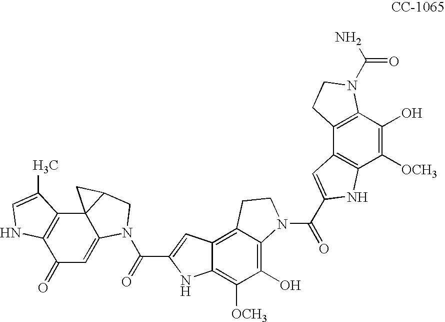

13. The system of claim 1, wherein the bioactive agent comprises the following molecule, or an analog or derivative thereof, or a pharmaceutically acceptable salt thereof:

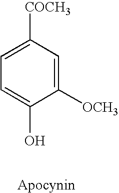

14. The system of claim 1, wherein the bioactive agent comprises at least one of the following molecules, or an analog or derivative thereof, or a pharmaceutically acceptable salt thereof:

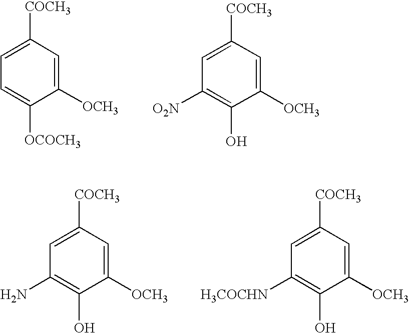

15. The system of claim 1, wherein the bioactive agent comprises the following molecule, or an analog or derivative thereof, or a pharmaceutically acceptable salt thereof: 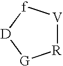
RGDfV, R: Arginine; G: Glycine; D: Aspartic acid; f: D-Phenylalanine; V: Valine

16. The system of claim 1, wherein the bioactive agent comprises the following molecule, or an analog or derivative thereof, or a pharmaceutically acceptable salt thereof:

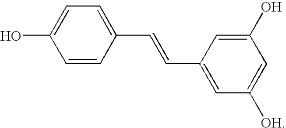

17. The system of claim 1, wherein the bioactive agent comprises the following molecule, or an analog or derivative thereof, or a pharmaceutically acceptable salt thereof:
Long chain unsaturated fatty acid-linker-CPT (Formula I);
wherein:
the Long-chain unsaturated fatty acid is generally C₁₂-C₂₂ mono or poly unsaturated fatty acids, which include, but are not limited to, palmitoleic acid, oleic acid, linoleic acid, linolenic acid, arachidonic acid, eicosapentaenoic acid (EPA) and docosahexaenoic acid (DHA);
CPT is a camptothecin compound with the following general structure (Formula II): 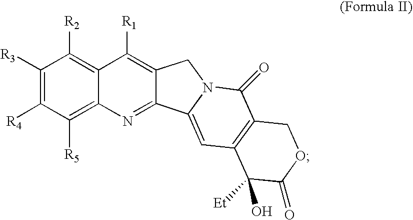
R₁-R₅ are H, halo, OH, NO₂, NH₂, alkyl, O-alkyl, NH-alkyl, N(alkyl)₂, and can be the same or different;
when any of R₁-R₅ is amino, the compounds are the free bases and their acid addition salts, such as HCl and H₂SO₄; and
the linker is selected from formula (III):

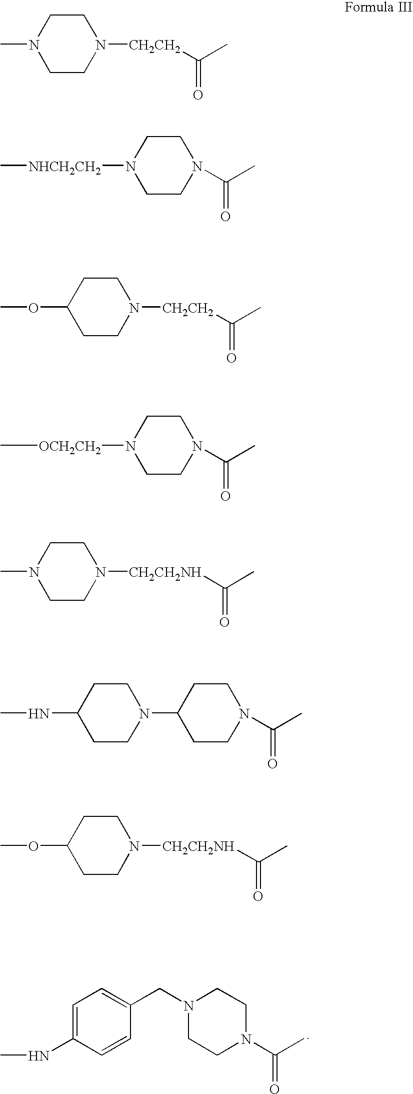

18. The system of claim 1, wherein the bioactive agent comprises at least one of the following molecules, or an analog or derivative thereof, or a pharmaceutically acceptable salt thereof:

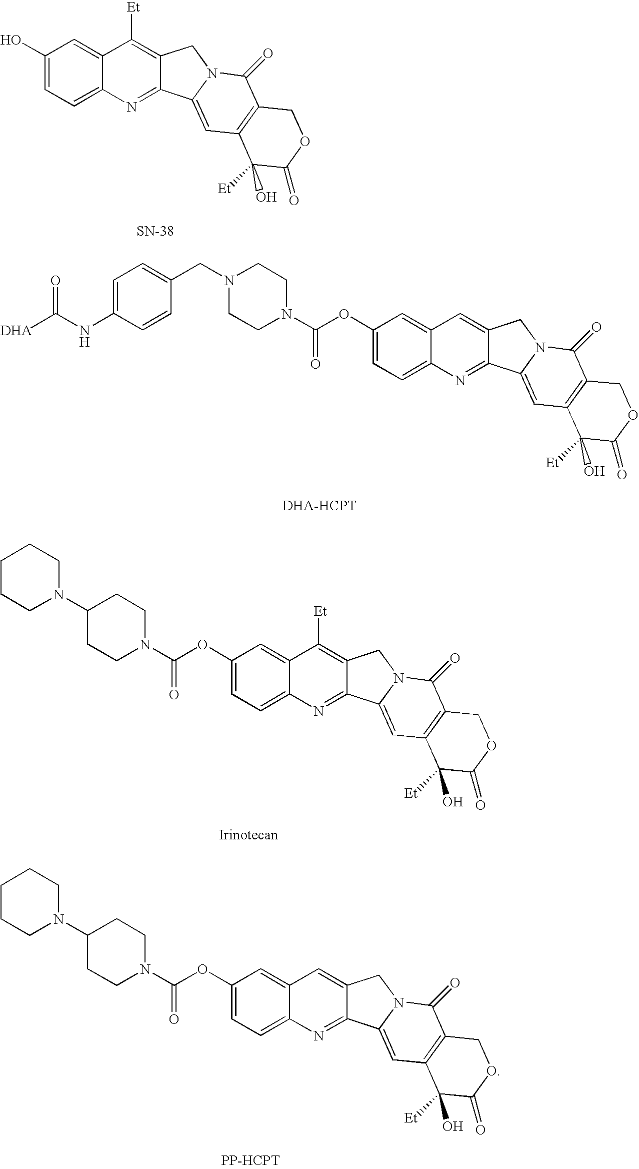

19. The system of claim 1, wherein the bioactive agent comprises at least one of the following molecules, or an analog or derivative thereof, or a pharmaceutically acceptable salt thereof:

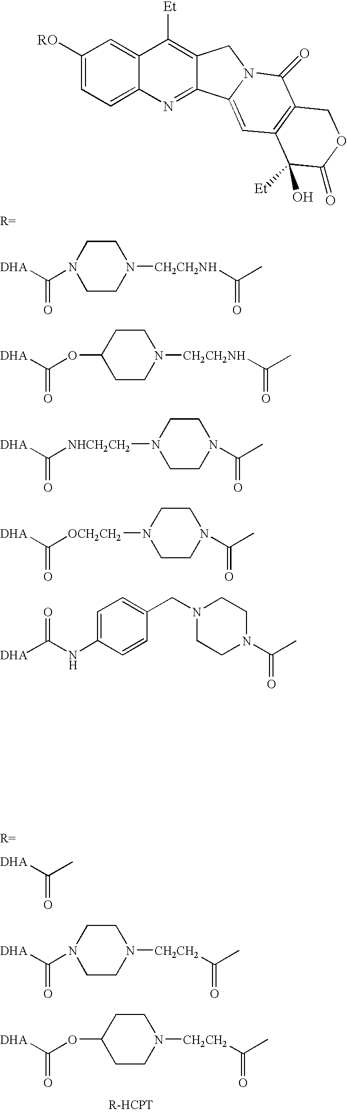

20. The system of claim 1, wherein the bioactive agent comprises a molecule having substantially the following amino acid sequence of SEQ ID NO:1, or an analog or derivative or conservative substitution variant thereof.

21. The system of claim 1, wherein the bioactive agent comprises the following molecule having the following amino acid sequence of SEQ ID NO:2, or an analog or derivative or conservative substitution variant thereof.

22. The system of claim 1, wherein the bioactive agent comprises the following molecule having the following amino acid sequence of SEQ ID NO:3, or an analog or derivative or conservative substitution variant thereof.

23. The system of claim 1, wherein the bioactive agent comprises one or more of the following molecules, or an analog or derivative thereof, or a pharmaceutically acceptable salt thereof:

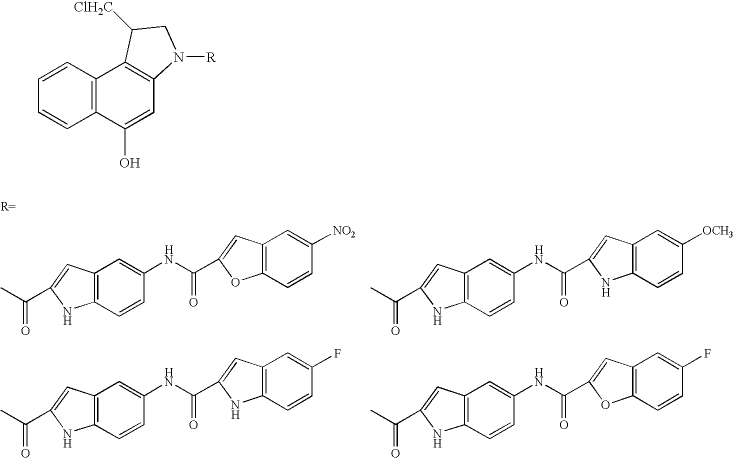

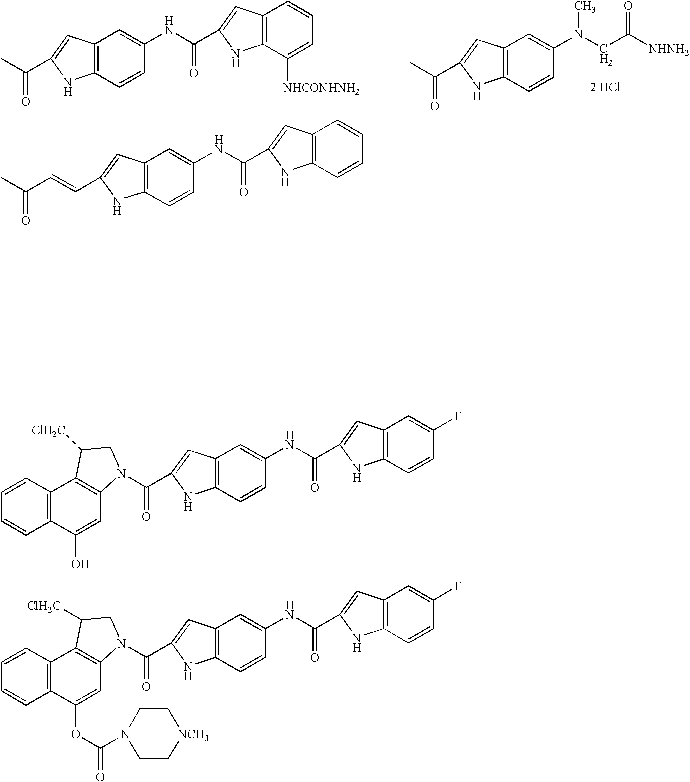

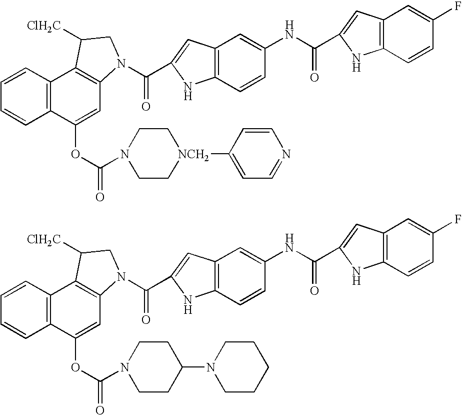

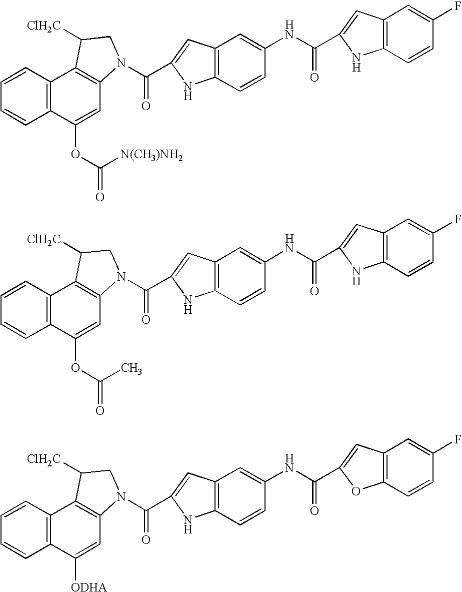

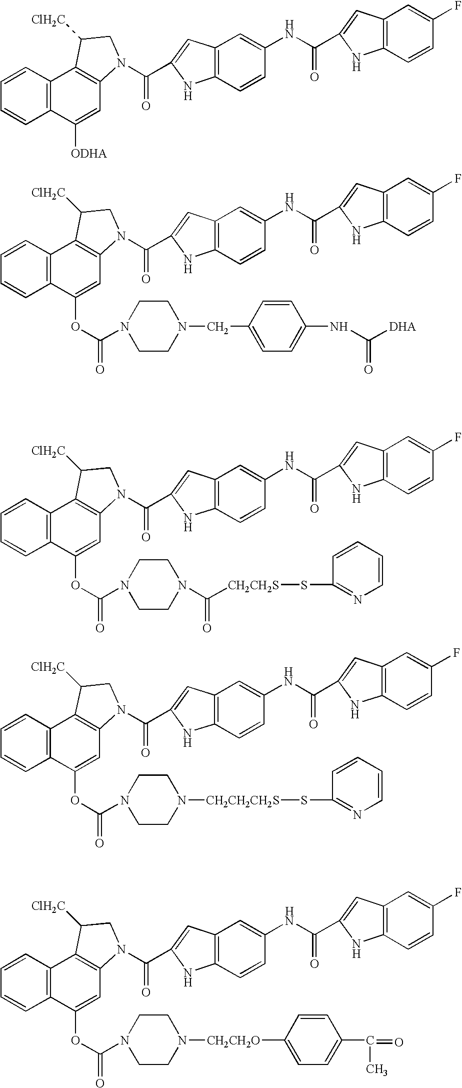

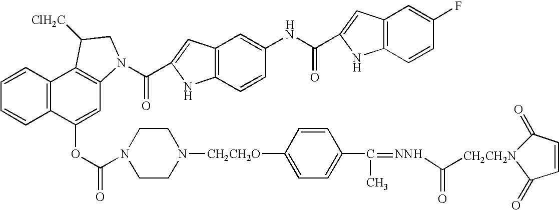

24. The system of claim 1, wherein the system further comprises:
an international medical device that is adapted to perform a medical procedure at a location associated with the region of tissue.

25. The system of claim 23, wherein the interventional medical device comprises an implantable stent.

26. The system of claim 24, wherein the local delivery system comprises a coating on the stent.

27. A method for treating or preventing atherosclerosis, stenosis, restenosis, smooth muscle cell proliferation, occlusive disease, or other abnormal lumenal cellular proliferation condition within a body of a patient, comprising:
locally delivering a bioactive agent at a location within the patient's body;
wherein the bioactive agent is locally delivered at the location in a manner that is adapted to substantially treat or prevent the atherosclerosis, stenosis, restenosis, smooth muscle cell proliferation, occlusive disease, or other abnormal lumenal cellular proliferation condition; and
wherein the bioactive agent comprises at least one of CC-1065, duocarmycin, apocynin, RGDfV, RGD peptide, resveratrol, a stilbene compound, camptothecin, des-aspartate angiotensin I (“DAA-1”), or apoptosis DNA factor (“ADF”), or an analog or derivative thereof, or a pharmaceutically acceptable salt thereof, or a combination or blend thereof.

28. The method of claim 27, further comprising:
injuring a wall of a lumen in the patients body; and
wherein the bioactive agent is locally delivered to the location in a manner adapted to substantially treat or prevent restenosis associated with the wall injury.

29. The method of claim 27, further comprising:
implanting a stent at the location.

30. The method of claim 29, further comprising:
eluting the bioactive agent from the stent at the location.
